(19) United States
(12) Patent Application Publication
(10) Pub. No.: US 2015/0313532 A1
(43) Pub. Date: Nov. 5, 2015

(54) METHOD AND SYSTEM FOR MANAGING AND QUANTIFYING SUN EXPOSURE

(71) Applicants: SAVA MARINKOVICH, JERSEY CITY, NJ (US); RAHUL MEHENDALE, JERSEY CITY, NJ (US); JADRAN BANDIC, PANCEVO (RS); DJURO KORUGA, BELGRADE (RS)

(72) Inventors: SAVA MARINKOVICH, JERSEY CITY, NJ (US); RAHUL MEHENDALE, JERSEY CITY, NJ (US); JADRAN BANDIC, PANCEVO (RS); DJURO KORUGA, BELGRADE (RS)

(21) Appl. No.: 14/799,687

(22) Filed: Jul. 15, 2015

Related U.S. Application Data
(63) Continuation-in-part of application No. 11/970,448, filed on Jan. 7, 2008.
Application No. 11/970,448 is a continuation-in-part of application No. 12/350,164, filed on Jan. 7, 2009, now abandoned.
Application No. 12/350,164 is a continuation-in-part of application No. 12/690,749, filed on Jan. 20, 2010.
Application No. 12/690,749 is a continuation-in-part of application No. 13/036,783, filed on Feb. 28, 2011.
(60) Provisional application No. 62/024,699, filed on Jul. 15, 2014.
(60) Provisional application No. 62/120,378, filed on Feb. 25, 2015.
(60) Provisional application No. 60/883,769, filed on Jan. 5, 2007.
(60) Provisional application No. 60/883,764, filed on Jan. 5, 2007.
(60) Provisional application No. 60/883,768, filed on Jan. 5, 2007.
(60) Provisional application No. 61/019,440, filed on Jan. 7, 2008.
(60) Provisional application No. 61/061,852, filed on Jun. 16, 2008.
(60) Provisional application No. 61/145,756, filed on Jan. 20, 2009.
(60) Provisional application No. 61/150,010, filed on Feb. 5, 2009.
(60) Provisional application No. 61/149,025, filed on Feb. 2, 2009.
(60) Provisional application No. 61/149,027, filed on Feb. 2, 2009.
(60) Provisional application No. 61/150,053, filed on Feb. 5, 2009.
(60) Provisional application No. 61/150,331, filed on Feb. 6, 2009.
(60) Provisional application No. 61/169,316, filed on Apr. 15, 2009.
(60) Provisional application No. 61/235,362, filed on Aug. 20, 2009.
(60) Provisional application No. 61/254,214, filed on Oct. 23, 2009.
(60) Provisional application No. 61/310,287, filed on Mar. 4, 2010.
(60) Provisional application No. 61/308,704, filed on Feb. 26, 2010.
(60) Provisional application No. 61/332,413, filed on May 7, 2010.
(60) Provisional application No. 61/380,003, filed on Sep. 3, 2010.
(60) Provisional application No. 61/386,962, filed on Sep. 27, 2010.
(60) Provisional application No. 61/407,454, filed on Oct. 28, 2010.
(60) Provisional application No. 61/380,155, filed on Sep. 3, 2010.
(60) Provisional application No. 61/431,926, filed on Jan. 12, 2011.

(51) Int. Cl. A61B 5/00; A61B 5/103

(52) U.S. Cl. CPC A61B 5/445; A61B 5/1032; A61B 5/0064; A61B 5/486; A61B 5/443; A61B 5/0075; A61B 5/6898; A61B 5/0013

(57) ABSTRACT

Embodiments of the present invention disclose a method and system for managing pre- and in-sun exposure of skin. The method comprises during pre-sun exposure phase, inputting a location information of a user, and other contextual ambient information therefor, thereby facilitating determination of the UV level thereof, determining a baseline information in connection with the skin in a part of the day from at least one of midnight and dawn to noon, scanning at least one most predictably susceptible zone of one or more zones of the skin predictably susceptible to sun exposure, determining the Sun Protection Factor (SPF) level for a sunscreen for use by the user based on one or more quantifiable qualitative and quantitative parameters therefor, recommending the determined SPF level for the sunscreen for use by the user thereby facilitating search, identification and selection of one or more sunscreen products, solutions and regimens by the user, during in-sun exposure phase, at least one of determining whether or not the UV index of the location is confined in at least one of one or more predetermined ranges and reminding the user to scan the skin at one or more predetermined intervals depending upon the confinement of the determined UV index or level in at least one of the one or more predetermined ranges.

### CROSS-REFERENCE TO RELATED APPLICATIONS

This application claims benefit of the following U.S. Provisional Applications, each of which is incorporated herein by reference in its entirety: U.S. Provisional Patent Application No. 62/024,699 filed Jul. 15, 2014 and U.S. Provisional Patent Application No. 62/120,378 filed Feb. 25, 2015.

This application is a continuation-in-part of the following U.S. Non-Provisional application, which is hereby incorporated by reference in its entirety: U.S. Non-Provisional patent application Ser. No. 11/970,448, filed Jan. 7, 2008, which claims the benefit of the following U.S. Provisional Applications, each of which is hereby incorporated by reference in their entirety: U.S. Provisional Patent Application No. 60/883,769, filed Jan. 5, 2007; U.S. Provisional Patent Application No. 60/883,764, filed Jan. 5, 2007; and U.S. Provisional Patent Application No. 60/883,768, filed Jan. 5, 2007.

This application is a continuation-in-part application of the following U.S. Non-Provisional application, which is hereby incorporated by reference in its entirety: U.S. Non-Provisional patent application Ser. No. 12/350,164, filed Jan. 7, 2009, which claims the benefit of the following U.S. Provisional Applications, each of which is hereby incorporated by reference in their entirety: U.S. Provisional Patent Application No. 61/019,440, filed Jan. 7, 2008 and U.S. Provisional Patent Application No. 61/061,852, filed Jun. 16, 2008.

This application is a continuation-in-part of U.S. Non-Provisional patent application Ser. No. 12/690,749, filed Jan. 20, 2010, which is incorporated herein by reference in its entirety and which claims the benefit of the following U.S. Provisional applications, each of which is hereby incorporated by reference in its entirety: U.S. Provisional Patent Application No. 61/145,756, filed Jan. 20, 2009; U.S. Provisional Patent Application No. 61/150,010, filed Feb. 5, 2009; U.S. Provisional Patent Application No. 61/149,025, filed Feb. 2, 2009; U.S. Provisional Patent Application No. 61/149,027, filed Feb. 2, 2009; U.S. Provisional Patent Application No. 61/150,053, filed Feb. 5, 2009; U.S. Provisional Patent Application No. 61/150,331, filed Feb. 6, 2009; U.S. Provisional Patent Application No. 61/169,316, filed Apr. 15, 2009; U.S. Provisional Patent Application No. 61/235,362, filed Aug. 20, 2009; and U.S. Provisional Patent Application No. 61/254,214, filed Oct. 23, 2009.

This application is a continuation-in-part of U.S. Non-Provisional patent application Ser. No. 13/036,783 filed Feb. 28, 2011, which is incorporated herein by reference in its entirety and which claims the benefit of the following U.S. Provisional Applications, each of which is hereby incorporated by reference in its entirety: U.S. Provisional Patent Application No. 61/310,287, filed Mar. 4, 2010; U.S. Provisional Patent Application No. 61/308,704 filed Feb. 26, 2010; U.S. Provisional Patent Application No. 61/332,413 filed May 7, 2010; U.S. Provisional Patent Application No. 61/380,003 filed Sep. 3, 2010; U.S. Provisional Patent Application No. 61/386,962 filed Sep. 27, 2010; U.S. Provisional Patent Application No. 61/407,454 filed Oct. 28, 2010; U.S. Provisional Patent Application No. 61/380,155 filed Sep. 3, 2010; and U.S. Provisional Patent Application No. 61/431,926 filed Jan. 12, 2011.

### BACKGROUND OF THE INVENTION

1. Field of the Invention

Embodiments of the present invention generally relate to health effects of sun exposure, and more particularly, to a method and system for managing and quantifying sun exposure.

2. Description of the Related Art

The Ultraviolet (UV) radiation in sunlight may be a principal source of vitamin D3 comparable to diet. Specifically, Ultraviolet (UV) B radiation with a wavelength of 290-315 nanometers may penetrate uncovered skin and convert cutaneous 7-dehydrocholesterol to previtamin D3, which in turn may become vitamin D3. However the UV radiation may be mutagenic, for example by virtue of the mutagenic effect of the UV radiation on skin.

Supplementing diet with vitamin D3 may supply vitamin D without the mutagenic effect. However, vitamin D3 dietary supplement may bypass natural mechanisms that prevent overdoses of vitamin D generated internally from sunlight.

Sunlight is the major source of vitamin D-producing UV B radiation, which may have a wide range of positive health effects including, but not limited to, skin and bodily health benefits, potential reduction in the growth of some cancers.

For example, a study in the Journal of the National Cancer Institute (NCI) looked at markers of sun exposure in more than 500 people, who had recently been diagnosed with malignant melanoma. Based on the findings of the study, solar elastosis, or sun damage to the skin, was independently associated with a surprising increased survival from melanoma. All measures of sun exposure, for example history of severe sunburn, high levels of intermittent sun exposure, solar elastosis, were associated with improved survival from melanoma.

On the other hand, at least one of long-term exposure and short-term over exposure may cause permanent skin damage and accelerates the onset of skin diseases including, but not limited to, skin cancer, acne, skin aging.

Despite the importance of the sun to vitamin D synthesis, it is prudent to limit exposure of skin to sunlight and UV radiation from tanning beds. According to the facts and figures of National Toxicology Program (NTP) report on carcinogens by the Department of Health and Human Services (HHS), broad-spectrum UV radiation is a carcinogen thought to contribute to most of the estimated 1.5 million skin cancers and the 8,000 deaths due to metastatic melanoma that occur annually in the United States. Lifetime cumulative UV damage to skin may also be largely responsible for some age-associated dryness and other cosmetic changes.

Hitherto, whether or not a desirable level of regular sun exposure exists that imposes at least one of no and minimal risk of skin cancer over time is not known. According to a study submitted by the University of Ottawa to the HHS, determination of a safe level of sun exposure at this time is a problem owing to lack of information. In addition, there is no consensus on which component of UV radiation (UVA or UVB or UVC) is actually carcinogenic, whilst the composition of UV radiation changes throughout the day. For example, at high noon, UV radiation reaching ground level is 95% UVA and 5% UVB, whereas before 10 AM and after 2 PM the percentages of UVA and UVB correspondingly changes over time to 99% UVA and 1% UVB.

Furthermore, there are currently no recommendations on the total safe level of lifetime sun exposure. Another detrimental effect of UV exposure is accelerated skin aging, also called skin photodamage, which produces a cosmetic effect that is difficult to treat. Thus, the American Academy of Dermatology (AAD) advises that photoprotective measures be taken, including the use of sunscreen, whenever one is exposed to the sun.

Season, geographic latitude, time of day, cloud cover, smog, skin melanin content, and sunscreen are among the factors that affect UV radiation exposure and vitamin D synthesis. Sunscreen, also commonly known as sunblock, sun screen, suntan lotion, sunburn cream, sun cream or block out, is a lotion, spray, gel or other topical product that absorbs or reflects some of the sun's UV radiation on the skin exposed to sunlight and thus helps protect against sunburn. Thus, depending on the mode of action responsive to sunlight, sunscreens may be classified into physical sunscreens, i.e., those that reflect the sunlight, and chemical sunscreens, i.e., those that absorb the UV light. Use of proper sunscreen cream or lotion or by gradually building up melanocytes with increasing exposure results in avoidance of sunburn, which can have mild to severe inflammation effects on skin. Besides, sunscreen is widely agreed to prevent sunburn and some types of skin cancer.

Medical organizations, such as the American Cancer Society (ACS), recommend the use of sunscreen because the sunscreen aids in the prevention of development of melanoma, squamous cell carcinomas and basal-cell carcinomas. In addition, the sunscreen is effective in preventing basal cell carcinoma. For example, in 2008, a clinical study showed that the application of sunscreen prevents squamous cell carcinoma, basal cell carcinoma, and actinic keratosis.

Many sunscreens do not block UVA radiation, which does not cause sunburn but can increase the rate of melanoma and photodermatitis, so people using sunscreens may be exposed to high UVA levels without realizing it. Incomplete protection against the full ultraviolet spectrum, combined with increased time spent in the sun, can lead to an increase in the risks of developing malignant melanoma, a rarer but more deadly form of skin cancer. The use of broad-spectrum (UVA/UVB) sunscreens can address this concern. Diligent use of sunscreen can also slow or temporarily prevent the development of wrinkles and sagging skin. For example, a study concluded that the diligent, everyday application of sunscreen can slow or temporarily prevent the development of wrinkles and sagging skin. The study involved 900 white people in Australia and required some of them to apply a broad-spectrum sunscreen every day for four and a half years. Based on the findings of the study, people who practiced the aforementioned regimen had noticeably more resilient and smoother skin than those assigned to continue the usual practices.

Sunscreens with a Sun Protection Factor (SPF) of 8 or more appear to block vitamin D-producing UV rays, although in practice people generally do not apply sufficient amounts, cover all sun-exposed skin, or reapply sunscreen regularly. Skin likely synthesizes some vitamin D even when it is protected by sunscreen as typically applied.

Malignant melanoma has been found more frequently in sunscreen users compared to non-users in some studies. Other studies found fair skinned people used more sunscreen and had more skin cancer, but did not address cause and effect. However, a meta-analysis of 9067 patients from 11 case-control studies found no association between sunscreen use and development of malignant melanoma. It was suggested that sunscreens block the natural warnings and adaptations mediated by UVB, but allow damage from UVA to go unchecked. However, the aforementioned claims could not be supported in three meta-analyses, namely 1) Huncharek M and Kupelnick B published in July 2002 and captioned “Use of topical sunscreens and the risk of malignant melanoma: a meta-analysis of 9067 patients from 11 case-control studies”; 2) Dennis L K, Beane Freeman L E and VanBeek M J published in December 2003 and captioned “Sunscreen use and the risk for melanoma: a quantitative review”; and 3) Gorham E D, Mohr S B, Garland C F, Chaplin G and Garland F C published in December 2007 and captioned “Do sunscreens increase risk of melanoma in populations residing at higher latitudes?”

The only evidence suggesting a relationship between sunscreen and melanoma is based on correlation, and thus cannot be used to establish a causal relationship.

Further, as per the speculations of authors claiming sunscreen use causes melanoma, melanoma occurs by one of the following mechanisms: 1) the absence of UVA filters combined with a longer exposure time of the sunscreen user; 2) by reducing the exposure of the skin to UVB radiation, sunscreen suppresses the skin's production of the natural photoprotectant, melanin, and the lack of melanin leads to an increased risk of melanoma; 3) free radical generation by sunscreen chemicals that have penetrated into the skin; pathogenic cytotoxicity and carcinogenicity of micronized titanium or zinc oxide nano-particles; and 4) retinyl palmitate, a form of vitamin A that is an ingredient in some sunscreens, may encourage tumor growth in animals. Diisopropyl adipate and retinoic acid have also demonstrated carcinogenic effects in mice.

Excessive exposure to direct sunlight is potentially harmful. Excessive exposure can result in sunburn if a person does not wear sun protective clothing or use suitable sunscreen. Products with a higher SPF (Sun Protection Factor) level provide greater protection against ultraviolet radiation.

Thus, there is still a need for a method and system for managing and quantifying sun exposure based on one or more qualitative and quantitative sun exposure management parameters. Specifically, there is still a need for a method and system for managing and quantifying sun exposure of the skin via at least one of measurement, determination and a combination thereof, one or more qualitative and quantitative skin parameters thereby facilitating quantification of quantitative sun exposure management parameters, determination of qualitative sun exposure management parameters, recommendation of optimal sun exposure management parameters for the skin both in the pre- and post- (or in-) sun exposure phases.

### SUMMARY OF THE INVENTION

Embodiments of the present invention generally relate to managing skin health, and more particularly, to imaging, analyzing, quantifying and classifying skin acne and recommending therapies and tracking efficacy of the recommended therapies.

These and other systems, processes, methods, objects, features, and advantages of the present invention will be apparent to those skilled in the art from the following detailed description of the preferred embodiment and the drawings. All documents mentioned herein are hereby incorporated in their entirety by reference.

Real-time analysis of digitally captured skin characteristics facilitates timely skin condition assessment, skin regimen recommendation, and skin regimen effectiveness tracking.

The problem of generating a skin condition assessment in real-time is solved by having a skin condition analysis module capable of doing real-time analysis of digital skin data, acquired partly using diffused reflectance spectroscopy and/or detecting the red-green-blue components of re-emitted white light.

In an aspect of the invention, a skin care device may include an electromagnetic radiation source capable of directing incident electromagnetic radiation to a location on the skin of a user, a radiation detector for measuring various parameters of radiation re-emitted from the location, and a skin condition analysis module coupled to the detector, the analysis module capable of generating a skin condition assessment in real-time, based partly on at least one of RGB analysis and diffused reflectance analysis of the radiation parameters. In the device, the incident electromagnetic radiation may include radiation in at least one of the visible, near-infrared, and near-ultraviolet spectrum. The incident radiation may include white light. In the device, the radiation parameters may include at least the degree of polarization of the re-emitted radiation. In the device, the radiation source may be a set of light emitting diodes. In the device, the skin condition assessment may also be partly based on analysis of a photographic image of a skin region surrounding the location. In the device, the device may be a miniature device. Miniature may mean that no dimension of the detector exceeds six inches. The device may further comprise a memory module for storing the skin condition assessment. The device may further comprise a user interface. The user interface may be operated using voice commands. In the device, skin assessment data of locations may be overlaid on an image of a larger skin region and displayed on the display surface. The device may further comprise an access restriction module used for restricting access to authorized users only. The access restriction module may be based on biometric access control. The device may be capable of generating alerts about abnormal skin conditions in real-time. The device may further comprise a skin care regimen recommendation module that generates a displayable skin care regimen recommendation. The skin care regimen recommendation may be based at least partly on determination of a skin profile of the user and use of skin care regimen recommendations of persons with a similar profile. The skin care regimen recommendation module may be linked to a product database. The product database may include products available in a point-of-sale location. The availability of a specific product recommended by the skin care regimen recommendation module may be indicated by an audio-visual signal. The device may further comprise a skin care regimen effectiveness module that generates a displayable skin care regimen effectiveness report. The device may further comprise a communication module for communicating with a remote computer. The communication may occur wirelessly. The communication may occur over an internet. The remote computer may be operable by a physician. The device may be wand-shaped. The device may be wearable by the user.

In an aspect of the invention, the skin care device may include an electromagnetic radiation source capable of directing incident electromagnetic radiation to a location on the skin of a user, a detector for measuring various parameters of radiation re-emitted from the location, a skin condition analysis module coupled to the detector, the analysis module capable of generating a skin condition assessment in real-time, based partly on at least one of RGB analysis and diffused reflectance analysis of the radiation parameters, and a display panel for reflecting the image of the user. In the device, the display panel may be touch-sensitive such that touching the location in a skin region image displayed in the display panel triggers display of a magnified image of the location. The device may further comprise a camera. The camera may be integral with the display panel. The camera may be wirelessly linked to the display panel. In the device, the display panel may be a mirror. In the device, a stored image of the user is used to automatically identify the person. The device may further comprise a user interface for controlling the skin care device. The user interface may be operated using voice commands. The device may further comprise a skin care regimen recommendation module capable of generating a displayable skin care regimen recommendation. The skin care regimen recommendation may be based at least partly on determination of a skin profile of the user and use of skin care regimen recommendations of persons with a similar profile. The device may further comprise a skin care regimen effectiveness module capable of generating a displayable skin care regimen effectiveness report.

In aspects of the invention, an imaging device permits a user to take high magnification pictures of the skin in the vicinity of an area of concern and submit those pictures, optionally along with textual and data responses, for medical, non-medical, and cosmetic analysis, diagnosis and treatment recommendation and follow-up.

In an aspect of the invention, a method and system of a non-invasive imaging device may comprise an illumination source comprising an incident light source to direct light upon skin; and a detector for detecting the degree of polarization of light reflected from the skin. In the method and system, the illumination source may be positioned to direct light at a selected angle alpha. Varying alpha may vary the depth of the measurement of the layers in the skin. Each depth may have a specific angle which produces a full polarized reflection. In the method and system, the incident light source may be an unpolarized light source. The unpolarized light may be white light, multiple selected wavelengths, or a single wavelength. The method and system may further comprise a sensor for capturing an image of the reflected or re-emitted light. The method and system may further comprise an optical facility for detecting reflected or re-emitted light from the skin. The method may determine both reflected or re-emitted light, and newly emitted light, through the process of absorption and re-emission. The method and system may further comprise a communication facility for transmitting the detected information. The method and system may further comprise a storage facility for storing information collected by the device.

In an aspect of the invention, a method and system for determining a skin state may comprise illuminating skin with an incident light source, detecting the degree of polarization of light reflected from the skin, and determining a skin state based on an aspect of the polarization of the reflected or re-emitted light. In the method and system, the incident light may be directed at a selected angle alpha. Varying alpha may vary the depth of the measurement of the layers in the skin. Each depth may have a specific angle which produces a full polarized reflection. In the method and system, the incident light source may be an unpolarized light source. The unpolarized light may be white light, multiple selected wavelengths, or a single wavelength. In the method of claim, the aspect of the polarization may be at least one of an orientation, an amplitude, a phase, an angle, a shape, a degree, an amount, and the like. In the method and system, determining may be done using an algorithm. The algorithm may involve artificial neural networks, non-linear regression, genetic algorithms, fuzzy logic, fractal and multi-fractal analysis, and the like. The methods and systems may further comprise filtering the reflected or re-emitted light to obtain polarized light of at least one wavelength defined by the filter output. The algorithmic analysis may be performed on the filtered image. In the method and system, determining may involve creating an image from the difference between the reflected diffusion light and the reflected polarized light. In the method and system, determining may involve comparing the aspect of the polarization of the reflected or re-emitted light to a calibration signal. In the method and system, determining may further comprise considering at least one of user input and a visual analysis.

In an aspect of the invention, a non-invasive imaging device may comprise an illumination source comprising an incident light source to direct light upon an area of concern and a detector for detecting the degree of polarization of light reflected from the area of concern. In the method and system, the illumination source may be positioned to direct light at a selected angle alpha. Varying alpha may vary the depth of the measurement of the layers in the skin. Each depth may have a specific angle which produces a full polarized reflection. In the method and system, the incident light source may be an unpolarized light source. The unpolarized light may be white light, multiple selected wavelengths, or a single wavelength. The method and system may further comprise a sensor for capturing an image of the reflected or re-emitted light. The method and system may further comprise an optical facility for detecting reflected or re-emitted light from the skin. The method and system may further comprise a communication facility for transmitting the detected information. The method and system may further comprise a storage facility for storing information collected by the device.

In an aspect of the invention, a method of determining moisture levels in the skin may comprise emitting incident light towards a skin structure, detecting a degree of polarization of the light induced by the skin structure, and determining a moisture level based on the amount of polarized and reflected or re-emitted light. The method and system may further comprise combining the assessment of moisture level with skin color measurements to determine luminosity. In the method and system, the incident light may be unpolarized light. The unpolarized light may be white light, light of multiple selected wavelengths, or of a single wavelength, or one or more monochromatic lights. In the method and system, determining may involve use of an algorithm. In the method and system, determining a moisture level may be based on the ratio of polarized and reflected or re-emitted light.

In an aspect of the invention, a method and system of determining elasticity of the skin may comprise emitting incident light towards a skin structure, detecting an aspect of polarization of the light reflected by the skin structure, correlating the aspect of polarization with a concentration of elastin, and determining elasticity level based on the elastin status. In the method and system, determining may involve use of an algorithm. In the method and system, the incident light may be unpolarized light. The unpolarized light may be white light, light of multiple selected wavelengths, or a single wavelength of light.

In an aspect of the invention, a method and system of determining firmness of the skin may comprise emitting incident light towards a skin structure, detecting an aspect of polarization of the light reflected by the skin structure, correlating the aspect of polarization with the status of at least one of an elastin, a collagen, and an activity of a sebaceous gland, and determining firmness based on the concentration of at least one of elastin and collagen and sebaceous gland activity. In the method and system, the sebaceous gland activity may be indicated by at least one of a number of glands, percent of glands open/closed, and level of clog/fill. In the method and system, correlating may involve use of an algorithm.

In an aspect of the invention, a method and system for obtaining dermal biophysical properties may comprise performing a spectral analysis of image data acquired from the degree of polarization of reflections and absorption and re-emission of incident light from skin structures, wherein the property is at least one of a structure, form, status, number, size, state, and stage of at least one of a: melanocyte, melanin, hemoglobin, porphyrin, triptofan, NADH, FAD, keratin, carotene, collagen, elastin, sebum, sebaceous gland activity, pore (sweat and sebaceous), moisture level, elasticity, luminosity, firmness, fine line, wrinkle count and stage, pore size, percent of open pores, skin elasticity, skin tension line, spot, skin color, psoriasis, allergy, red area, general skin disorder or infection, tumor, sunburn, rash, scratch, pimple, acne, strias, insect bite, itch, bleeding, injury, inflammation, photodamage, pigmentation, tone, tattoo, percent burn/burn classification, mole (naevi, nevus), aspect of a skin lesion (structure, color, dimensions/asymmetry), melanoma, automated follow-up of pigmented skin lesions, dermally observed disorder, cutaneous lesion, cellulite, boil, blistering disease, congenital dermal syndrome, (sub)-cutaneous mycoses, melasma, vascular condition, rosacea, spider vein, texture, skin ulcer, wound healing, post-operative tracking, melanocytic lesion, non-melanocytic lesion, basal cell carcinoma, seborrhoic keratosis, sebum (oiliness), nail- and/or hair-related concern, and the like.

In an aspect of the invention, a system and method may comprise providing an interface that includes a social networking domain or rating-and-ranking system and at least one of a skin state determination facility and a recommendation engine, and enabling users, either all or a selected few, of the interface to perform a skin state determination within the interface. In the method and system, the skin state determination facility may comprise capturing images with a non-invasive imaging device comprising an illumination source comprising an incident light source to direct light upon skin, and a detector for detecting the degree of polarization of light reflected from the skin, and determining a skin state based on an aspect of the polarization of the reflected or re-emitted light. The method and system may further comprise receiving product and regimen recommendations from the recommendation engine based on what other users with similar skin states are using as well as data regarding ingredients, effectiveness, safety, and the like. The method and system may further comprise comparing skin states, products, regimens, and recommended products or regimens with peers within the social networking domain of the interface. Comparing may comprise an analysis of similarity based on the spectral analysis of the degree of polarization of reflected or re-emitted light from users' skin. In the method and system, the interface may comprise a regimen tracker. The regimen tracker may be populated using a drag-and-drop or click-to-add functionality. In the method and system, the interface may comprise a rating facility or a product information facility. The product information facility may enable a user to obtain product information by search. Search may be a search of product identifiers, product ratings, drag-and-drop items, images, barcode scans, skin states, and profiles.

In an aspect of the invention, a method and system for determining a skin state may comprise obtaining the answers to a series of subjective questions regarding the skin, obtaining an objective skin analysis using a dermal imaging device, and combining the subjective and objective results algorithmically to obtain a skin state.

In an aspect of the invention, a system and method for providing recommendations for skin care based on a skin state and a skin care goal may comprise obtaining a skin state of an individual, categorizing the individual by skin state, and recommending products and regimens that are effective for other individuals of the category in achieving the skin care goal. In the method and system, the system may be operable over a network. In the method and system, the skin state may be determined based on analysis of the degree of polarization of light reflected from the skin of the individual.

In an aspect of the invention, a method for tracking the effectiveness of a skin care product or regimen may comprise obtaining a baseline skin state assessment, recommending a monitoring interval based on at least one of the skin care goal, product, and regimen, obtaining a second skin state assessment, comparing the second assessment to the baseline assessment to determine progress towards a skin care goal, and optionally, optimizing the regimen or product in order to improve a skin state. In the method and system, the skin assessment may be based on analysis of the degree of polarization of light reflected from the skin of the individual.

In an aspect of the invention, a personalized skin condition analysis system and related methods may comprise an imaging device, comprising an illumination source comprising an incident light source to direct light upon skin, and a detector for detecting the degree of polarization of light reflected from the skin, and a user interface for controlling the device. In the methods and system, the device may be adapted to interact with a physical interface to download image data to update a record of at least one of a practitioner, a spa, a salon, cosmetic sales, a cosmetics manufacturer, a clinical trials database, and a third party database. In the method and system, the illumination source may be positioned to direct light at a selected angle alpha. Varying alpha may vary the depth of the measurement of the layers in the skin. Each depth may have a specific angle which produces a full polarized reflection. In the method and system, the incident light source may be an unpolarized light source. The unpolarized light may be white light, multiple selected wavelengths, or a single wavelength. The method and system may further comprise a sensor for capturing an image of the reflected or re-emitted light. The method and system may further comprise an optical facility for detecting reflected or re-emitted light from the skin. The method and system may further comprise a communication facility for transmitting the detected information. The method and system may further comprise a storage facility for storing information collected by the device.

In an aspect of the invention, a non-invasive imaging device may comprise an illumination source comprising an incident light source to direct light upon skin; and a detector for detecting a characteristic of the light reflected from the skin. In the device, the illumination source may be positioned to direct light at a selected angle alpha. Varying alpha may vary the depth of the measurement of the layers in the skin. Each depth may have a specific angle which produces a full polarized reflection. In the device, the incident light source may be a polarized light source or unpolarized light source. The unpolarized light may be at least one of white light, light of a single wavelength, and light of multiple single wavelengths. The device may further comprise a sensor for capturing an image of the reflected or re-emitted light. The device may further comprise an optical facility for detecting reflected or re-emitted light from the skin. The device may further comprise a communication facility for transmitting the detected information. The device may further comprise a storage facility for storing information collected by the device. In the device, the reflected or re-emitted light may be at least one of polarized light and unpolarized light.

In an aspect of the invention, a method and system for determining a skin state may comprise illuminating skin with an incident light source; detecting a characteristic of the light reflected from the skin; and determining a skin state based on at least one characteristic of the reflected or re-emitted light. In the method and system, the incident light may be directed at a selected angle alpha. Varying alpha may vary the depth of the measurement of the layers in the skin. Each depth may have a specific angle which produces a full polarized reflection. In the method and system, the incident light may be unpolarized or polarized light. The unpolarized light may be at least one of white light, light of a single wavelength, and light of multiple single wavelengths. In the method and system, the reflected or re-emitted light may be at least one of polarized light and unpolarized light. In the method and system, the characteristic may be at least one of light source, light intensity, wavelength of light, angle of light, electrical and magnetic properties of the light, and polarization state of the light. An aspect of the polarization may be at least one of an orientation, an amplitude, a phase, an angle, a shape, a degree, and an amount. In the method and system, determining may be done using an algorithm. The algorithm may involve artificial neural networks, non-linear regression, genetic algorithms, fuzzy logic, or fractal and multi-fractal analysis. The method and system may further comprise filtering the reflected or re-emitted light to obtain light of a wavelength defined by the filter output. The analysis may be performed on the filtered image. In the method and system, determining may involve creating an image of the difference between reflected diffusion light and reflected polarized light. In the method and system, determining may involve comparing the aspect of the polarization of the reflected or re-emitted light to a calibration signal. In the method and system, determining may further comprise considering at least one of user input and a visual analysis.

In an aspect of the invention, a non-invasive imaging device may comprise an illumination source comprising an incident light source to direct light upon an area of concern; and a detector for detecting a characteristic of the light reflected from the area of concern. In the device, the illumination source may be positioned to direct light at a selected angle alpha. Varying alpha may vary the depth of the measurement of the layers in the skin. Each depth may have a specific angle which produces a full polarized reflection. In the device, the incident light source may be a polarized light source or unpolarized light source. The unpolarized light may be at least one of white light, light of a single wavelength, and light of multiple single wavelengths. The device may further comprise a sensor for capturing an image of the reflected or re-emitted light. The device may further comprise an optical facility for detecting reflected or re-emitted light from the skin. The device may further comprise a communication facility for transmitting the detected information. The device may further comprise a storage facility for storing information collected by the device. In the device, the reflected or re-emitted light may be at least one of polarized light and unpolarized light.

In an aspect the invention, a system and method may be used to determine healthy and melanocytic skin. The first, reflected spectrum and/or emission spectrum from sample which is skin malformation (SM), subtract reflected spectrum from normal healthy skin (SN). The second, from obtained resulting spectral plots (SM-SN) subtract reflected spectrum from adequate comparing screen, which represents spectral plot of the light source (SO). In that path appeared pure characteristics of change generated by skin. For differentiation between melanoma, other malignant or benign nevus and healthy skin can be used data on maxima, minima and zero positions, in wavelength scale and data on maxima and minima intensities.

In an aspect of the invention, a system and method may comprise capturing an image of a material illuminated with incident non-angled white light and angled white light, generating a normalized red and blue color channel histogram for each image, correlating the normalized red and blue color channel histograms to a wavelength scale to obtain red and blue color channel spectral plots, and convoluting the spectral plots by subtracting the spectral plot for angled light from the spectral plot for non-angled light for each color channel to generate red and blue normalized, composite color channel spectral plots, and subtracting the normalized, composite blue channel spectral plot from the normalized, composite red channel spectral plot to generate a spectral signature for the material. In the system and method, the illumination source may be positioned to direct light at a selected angle alpha. Varying alpha varies the depth of the measurement in the material. In the system and method, the unit scale on the spectral signature may be a difference of wavelength. In the system and method, the material is inorganic and/or organic matter. In the system and method, the spectral signature may be analyzed for at least one of number of peaks and troughs, amplitude and shape of peaks and intermediate structures and patterns. In the system and method, the spectral signature may be analyzed for metal composition, identification, purity, and strength. In the system and method, the spectral signature may be analyzed for water quality, composition, and purity. In the system and method, elements of the spectral signature may be tagged and tracked over time in order to track changes in the characteristics of the material. In the system and method, the spectral signature may be analyzed to measure, track or monitor a skin state. In the system and method, the spectral signature may be useful for the counterfeit analysis of money. In the system and method, the spectral signature may be analyzed for at least one of sweat gland activity and anti-perspirant effectiveness. In the system and method, the spectral signature may be analyzed for Mad Cow disease. In the system, the spectral signature may be analyzed for food, all epidermal diseases, melanoma and skin cancers, rheumatoid diseases, and all diseases that show on the skin. In the system and method, the spectral signature may be useful for monitoring post-operative cosmetic concerns. In the system and method, the spectral signature may be useful for predicting and monitoring secretion from the mammary glands of lactating women. In the system and method, the spectral signature may be fed into a recommendation engine to provide feedback and modifications to aspects of a regimen. In the system and method, the wavelength position of ideal blue in Maxwell's color triangle is aligned with the wavelength position of ideal red in Maxwell's color triangle when convoluting the composite spectral plots to obtain the spectral signature.

A method and a system are disclosed for determining skin characteristics and cosmetic features. A minimal error output is generated. In accordance with exemplary embodiments of the present invention, according to a first aspect of the present invention, a method for determining skin characteristics and cosmetic features using color analysis may include a step of analyzing color of skin images in a pixel by pixel manner in a Red Green Blue (RGB) color system for an acquired digital image. The step of analyzing color of skin images in a pixel by pixel manner in a RGB color system for an acquired digital image may include analyzing a picture of a part of a person's skin by generating a table of most frequent colors appearing in the picture.

According to the first aspect, a method for determining skin characteristics and cosmetic features using color analysis includes a step of generating a sample of most frequent standard RGB (sRGB) colors responsive to analyzing color of skin images in a pixel by pixel manner in the RGB color system for the acquired digital image after converting colors obtained in device dependent RGB color system into device independent standard RGB color system (sRGB). The step of generating a sample of most frequent sRGB colors responsive to analyzing color of skin images in the sRGB color system for the acquired digital image may include preserving a plurality of sRGB color values.

In this embodiment of the invention, the sRGB color system may be used for image analysis. Determination of other skin characteristics, melanoma, skin related tumors and skin related disorders require image analysis based on other color systems such as YIQ, YCbCr, L*a*b*, L*u*v* and HSL/HSV. The enhancement of the current algorithm may include at least one of these color systems and its/their correlation with presented sRGB analysis.

According to the first aspect, a method for determining skin characteristics and cosmetic features using color analysis includes a step of modeling the R, G and B component color distribution with Gaussian probabilistic distribution with estimated parameters (expected value and standard deviation) on the generated sRGB color sample for the acquired digital image further including approximating colors on the generated sRGB color samples by a Gaussian normal distribution. In accordance with an exemplary embodiment of the present invention the step of approximating colors on the generated sRGB color samples by a Gaussian normal distribution comprises approximating colors on the generated sRGB color samples by a superposition of a plurality of Gaussian normal distributions.

According to the first aspect, a method for determining skin characteristics and cosmetic features using color analysis includes a step of generating a phototype of the skin through a decision tree unit responsive to the estimated distribution model parameters colors. The phototype of the skin may be generated according to a corrected Fitzpatrick classification. In accordance with an exemplary embodiment of the present invention, the step of generating phototype of the skin according to corrected Fitzpatrick classification includes generating phototype of the skin according to a skin type scale which ranges from very fair skin to very dark skin. This method may be measured both on the most exposed region and relate to the current level of phototype based on level of tan on the skin.

According to a second aspect, a system for skin phototype determination using photograph analysis may be disclosed. The system may include an image capturing device for capturing digital images of a skin. The image capturing device may include a digital camera unit.

According to the second aspect, the system for skin phototype determination using photograph analysis may include an analyzer coupled to the image capturing device for performing a pixel by pixel analysis of a picture of a part of a person's skin. The analyzer may include a quantization device for generating a look-up table of most frequent colors appearing on the picture of the part of the person's skin.

According to the second aspect, the system for skin phototype determination using photograph analysis may include a sampling device coupled to the image capturing device for generating standard Red Green Blue (sRGB) color samples for the captured digital image of the skin.

According to the second aspect, the system for skin phototype determination using photograph analysis may include an approximating device coupled to the sampling device for approximating the color distribution parameters on the generated sRGB color samples using the estimates of expected value and standard deviation for the captured digital image of the skin. The approximating device may include at least one Gaussian normal distribution unit.

According to the second aspect, the system for skin phototype determination using photograph analysis may include a decision tree unit coupled to the approximating device for generating a phototype of the skin using Red and Blue components of the approximated colors. The decision tree unit may include a Fitzpatrick scaling unit for categorizing a skin phototype in accordance with a skin type scale which ranges from very fair skin to very dark skin.

According to the second aspect, an exemplary embodiment of the present invention discloses a scaled Gaussian normal distribution unit for approximating colors on the generated sRGB color samples using estimates of expected value and standard deviation for the captured digital image of the skin.

According to the second aspect of the present invention, the system for skin phototype determination using photograph analysis may include a subsystem for determination of cosmetic features for a human element and a veterinary element. The cosmetic features may further include features pertaining to hair, nail and skin.

In another aspect the system may include a sampling device for generating standard Red Green Blue color samples of the captured digital image of the skin, the generated samples of standard Red Green Blue are in the range of values between 0 and 255 and they are preserved for further processing.

In another aspect the system may include an approximating device coupled to the sampling device for approximating the color distribution parameters on the generated sRGB color samples in the range of values between 0 and 255 by Gaussian normal distribution using the estimates of expected value and standard deviation for the captured digital image of the skin.

In another aspect the system may further include a decision tree unit coupled to the approximating device for generating a phototype of the skin using standard Red and Blue components of the approximated colors, the decision tree unit with an algorithm equates estimates of expected values and standard deviation for the captured image of the skin to the Fitzpatrick notation of skin analysis for determination of skin phototype.

In another aspect the system may automatically adjust lighting intensity and wavelengths and angles in order to assess various factors of the skin.

In yet another aspect of the system skin phototype may be determined using photograph analysis for use in cosmetics and surgical industry.

In an aspect of the invention, a skin care device may include an electromagnetic radiation source capable of directing incident electromagnetic radiation to a location on the skin of a user, a radiation detector for measuring various parameters of radiation re-emitted from the location, and a skin condition analysis module coupled to the detector, the analysis module capable of generating a skin condition assessment in real-time, based partly on at least one of RGB analysis and diffused reflectance analysis of the radiation parameters. In the device, incident electromagnetic radiation may include radiation in at least one of the visible, near-infrared, and near-ultraviolet spectrum. The incident radiation may be white light. In the device, the radiation parameters include at least the degree of polarization of the re-emitted radiation. In the device, the radiation source may be a set of light emitting diodes. In the device, the skin condition assessment may be also partly based on analysis of a photographic image of a skin region surrounding the location. In the device, the device may be a miniature device. Miniature may mean that no dimension of the detector exceeds six inches. The device may further include a memory module for storing the skin condition assessment. The device may further include a user interface. The device may further include a display surface. The skin assessment data of locations may be overlaid on an image of a larger skin region and displayed on the display surface. The device may further include an access restriction module used for restricting access to authorized users only. The access restriction module may be based on biometric access control. The device may be capable of generating alerts about abnormal skin conditions in real-time. The user interface may be operated using voice and/or eye movement commands. The device may further include a skin care regimen recommendation module that generates a displayable skin care regimen recommendation. The skin care regimen recommendation may be based at least partly on determination of a skin profile of the user and use of skin care regimen recommendations of persons with a similar profile. The skin care regimen recommendation module may be linked to a product database. The product database may include products available in a point-of-sale location. The availability of a specific product recommended by the skin care regimen recommendation module may be indicated by an audio-visual signal. The device may further include a skin care regimen effectiveness module that generates a displayable skin care regimen effectiveness report. The device may further include a communication module for communicating with a remote computer. The communication may occur wirelessly. The communication may occur over an internet. The remote computer may be operable by a physician. The device may be wand-shaped. The device may be wearable by the user.

In an aspect of the invention, the device an electromagnetic radiation source capable of directing incident electromagnetic radiation to a location on the skin of a user, a detector for measuring various parameters of radiation re-emitted from the location, a skin condition analysis module coupled to the detector, the analysis module capable of generating a skin condition assessment in real-time, based partly on at least one of RGB analysis and diffused reflectance analysis of the radiation parameters, and a display panel for reflecting the image of the user. In the device, the display panel may be touch-sensitive such that touching the location in a skin region image displayed in the display panel triggers display of a magnified image of the location. The skin care device may further include a camera. The camera may be integral with the display panel. The camera may be wirelessly linked to the display panel. In the device, the display panel may be a mirror. In the device, a stored image of the user may be used to automatically identify the person. The device may further include a user interface for controlling the skin care device. The user interface may be operated using voice and/or eye movement commands. The device may further include a skin care regimen recommendation module capable of generating a displayable skin care regimen recommendation. The skin care regimen recommendation may be based at least partly on determination of a skin profile of the user and use of skin care regimen recommendations of persons with a similar profile. The device may further include a skin care regimen effectiveness module capable of generating a displayable skin care regimen effectiveness report.

In an aspect of the invention, a system and method for moving information objects available on a website to a receptacle to communicate with a plurality of people in a controlled access community network may include enabling movement of a plurality of information objects from a predetermined website to a web based network responsive to a regimen of a person, a routine of a person, a purpose of use of an information object of the plurality of information objects and a degree of affinity of a first person towards a second person, initiating at least one customized action from the actions including a drop down movement; a drag and drop movement for populating data; and a pop-up movement in a Graphical User Interface (GUI) responsive to enabling movement of a plurality of information objects from a predetermined healthcare website, and enabling transportation of the plurality of information objects across a plurality of websites. In the system and method, the plurality of information objects may pertain to a questionnaire on at least one of a human skin condition, product information, an article, a blog posting, an image, a video, an individual message, a forum posting, and a veterinary skin condition. In the system and method, the plurality of information objects pertains to a questionnaire on human cosmetic parameters and veterinary cosmetic parameters. The questionnaire on human cosmetic parameters and veterinary cosmetic parameters may include questions on at least one of a human nail and a veterinary nail. The questionnaire on human cosmetic parameters and veterinary cosmetic parameters may include questions on at least one of a human hair and a veterinary hair. In the system and method, the purpose of use of the information object may pertain to controlling at least one of cleansing, protection, repair, moisturizing, elasticity, firmness, glow, luminosity, anti-inflammatory properties, anti-itch properties, anti-wrinkle properties, firming, exfoliating, anti-redness properties, oil controlling, anti-aging properties and shine of a human skin. In the system and method, the degree of affinity of a first person towards a second person comprises at least one of a relationship of friendship between the first person and the second person; a genetic similarity between the first person and the second person; a similarity of lifestyle between the first person and the second person; a climatic similarity between a first residential environment and a second residential environment; and a skin type similarity between the first person and the second person. In the system and method, the step of enabling transportation of the plurality of information objects across a plurality of websites may include a sub-step of dragging an item of user interest off a website of the plurality of websites in a predetermined format and transferring through an electronic signal to affiliates of a user accessing the website. The affiliates of the user may be friends and relatives of the user or associated experts. In the system and method, the step of enabling movement of a plurality of information objects from a predetermined website to a web based network may include a sub-step of enabling drop down menus on the Graphical User Interface (GUI) responsive to a plurality of end user convenience and requirement parameters. In the system and device, the plurality of people in a web based network includes a plurality of people in an online friendship network. In the system and device, the plurality of people in a web based network includes a plurality of people in an online social network.

In an aspect of the invention, an interface including a social networking domain and at least one skin health assessment and recommendation unit for enabling users of the interface to perform a skin health assessment within the interface and to receive product and regimen recommendations from a recommendation engine based on a predetermined usage of health assessment and maintenance data may include a regimen tracker populated using a drag and drop facility, a rating unit for rating a plurality of healthcare facilities, and a product information unit for enabling a user to obtain product information by conducting a web based search of a plurality of web based drag and drop products, web based images and bar code scans. In the interface, the regimen tracker includes a diet tracking unit. In the interface, the plurality of healthcare facilities comprises at least one of skin cleansing, skin protection, skin moisture control, skin repair, skin elasticity, skin luminosity, skin firmness, skin wrinkles, pore size on skin, spots on skin, glow on skin, hair color, hair type, age and life stage further including marriage, pregnancy, dating and social life. In the interface, the product information comprises at least one of a product type, a product function, a product format, a product appropriateness level, a regimen information, product articles, product blogs, product safety, product toxicity, a product effectiveness index, a product cost information, and a product timeliness information. In the interface, the interface is a multiple language and customized interface for: web based applications; mobile phone applications; touch screen applications; and personal digital assistant applications. In the interface, the interface is seamlessly coupled with a dermal imaging device for customized web based access, control and maintenance of spectral analysis of image data acquired from a degree of polarization of reflections and re-emission of incident light from skin structures. The degree of polarization of reflections and/or re-emissions of incident light from skin structures is derived from at least one of a Red Green Blue (RGB) color analysis of a plurality of digital images; and an analysis from spectroscopic data image analysis.

In an aspect of the invention, a system and method for determining a health state may include obtaining the answers to a series of subjective questions regarding health conditions, obtaining an objective health assessment report through a dermal imaging device, and generating a combination of answers to the series of subjective questions and the objective health assessment report to thereby generate a health state output and a real skin type output. In the system and method, a real skin type output is generated based on biophysical properties generated by at least one of a person seeking skin health monitoring, a spa, and a cosmetic advisor. In the system and method, the objective health assessment report may include an objective skin health assessment report on at least one of systemic hydration, skin hydration, skin firmness, skin wrinkles, pore size on skin, spots on skin, glow on skin, melanocyte, melanin, hemoglobin, porphyrin, triptofan, NADH, FAH, keratin, carotene, collagen, elastin, sebum, sebaceous gland activity, sweat pore, sebaceous pore, moisture level, elasticity, luminosity, firmness, fine line, wrinkle count, pore size, percent of open pores, skin elasticity, skin tension line, spots, viscosity, epidermal, dermal sebum levels, skin color, psoriasis, allergy, red area, general skin disorder, infection, tumor, sunburn, rash, scratch, pimple, acne, insect bite, itch, bleeding, injury, inflammation, photodamage, pigmentation, tone, tattoo, percent burn, burn classification, mole, aspect of a skin lesion, melanoma, dermally observed disorder, cutaneous lesion, cellulite, strias, current tan level, boil, blistering disease, congenital dermal syndrome, cutaneous mycoses, melasma, vascular condition, rosacea, spider vein, texture, skin ulcer, wound healing, post-operative tracking, melanocytic lesion, nonmelanocytic lesion, basal cell carcinoma, seborrhoic keratosis, sebum hair color, hair type, nail condition, and age and life stage further including marriage, pregnancy, dating and social life. In the system and method, the objective health assessment report is sent to an end user through at least one of email, SMS, MMS, mobile phone, a graphical user interface (GUI) of an internet connected device, and a touch screen enabled personal digital assistant. The system and method may further include obtaining health assessment and maintenance data from a physiologically polarized light data. The step of obtaining health assessment and maintenance data from a physiologically polarized light data comprises obtaining health assessment and maintenance data from a Red Green Blue (RGB) color analysis device, wherein the data comprise at least one of a white light data, a blue light data, and an ultra violet light data. The step may further comprise obtaining at least one of the white light data, the blue light data, and the ultra violet light data by reading and recording conditions of at least one of the dermis and epidermis. Obtaining health assessment and maintenance data from a physiologically polarized light data comprises obtaining data pertaining to age, geography and demography for a person subjected to health monitoring.

In an aspect of the invention, a web-enabled health tracking method and system may include a camera comprising a photo guide unit for generating notes for each photograph captured, an interface coupled between the camera and a web-enabled computing system for uploading the photograph captured by the camera, a graphical user interface unit included in the web-enabled computing system for generating a frequently asked questionnaire unit further comprising a self answer guide module, a scoring module coupled to the frequently asked questionnaire unit, a comparison module coupled to the scoring module for comparing: a color parameter; a symmetry parameter; and a border parameter, an automation unit coupled to the graphical user interface for enabling a time-based synchronization of the frequently asked questionnaire unit, the scoring module, and the comparison module, and a learning unit coupled to the automation unit for activating: a user training module, an article module coupled to the user training module, a blogging unit coupled to the user training module and the article module, and a report unit including an email unit for emailing health related information. In the system and method, the camera comprises a tracking unit for tracking at least one of skin spots over time, laser treatment effectiveness, cellulite content in skin, current tan level, condition of veins and capillaries, botox treatment effectiveness, anti-aging treatment effectiveness, anti-acne treatment effectiveness, and a pictorial history of skin to be given to the doctor. The skin spots over time include at least one of blemishes, scars, rashes, lesions, and moles. In the system and method, the web-enabled computing system for uploading the photograph captured by the camera further includes a walkthrough module for walking through features of a skin health record of a first time user of the system, a personal skin photo album for reviewing pictorial history of a regular user of the system, and a product quality menu for tracking product expiration dates. In the system and method, the interface for uploading the photograph further includes a reminder unit for next photo for a regular user of the system; and a cosmetic status unit coupled to the reminder unit for displaying a current usage of a cosmetic for the regular user of the system. The current usage comprises a usage of at least one of a moisturizer, an antiseptic, a toner, a laser, and a botox. The system and method may further include a photo review unit for date based reviewing of at least one of a condition of a predetermined body part, a current usage status of a cosmetic, and a recommended usage list of cosmetics. In the system and method, the report unit may further include a secure transmission unit for sending a health assessment report to a medical practitioner, an affinity unit for discussing health assessment data with a friend, and a printing unit for printing health assessment data.

In an aspect of the invention, a mobile device-based health assessment system and method may include a photograph capturing device for capturing a skin image of a mobile device user, a transmission unit coupled with the photograph capturing device for uploading the captured skin image to a network location, a global positioning device coupled to the photograph capturing device for determining a location of the photograph capturing device, and a weather estimation device coupled to the photograph capturing device to determine a weather condition at a location of the mobile device user to thereby obtain a remote diagnosis report. In the system and method, the photograph capturing device further comprises at least one of a skin photograph assessment unit, a nail photograph assessment unit, and a hair photograph assessment unit. In the system and method, the global positioning device comprises a location tracker for answering user raised questions pertaining to geographical positioning of the user. In the system and method, the location tracker includes a database pertaining to weather intensive cosmetics. The system and method may further include a phone number tracker for enabling a mobile device user to contact health assessment and cosmetic outlets.

In an aspect of the invention, a system and method for estimation of skin type and skin features to create a unique spectral signature may include convoluting data from a first image captured in incident diffuse white light, wherein the data relate to reflected and/or re-emitted polarized or white light, convoluting data from a second image captured in incident polarized light, wherein the data relate to reflected and/or re-emitted polarized light, comparing extreme positions of at least two unique convolutions generated by convoluting data from the first image and the second image, and determining a distance between minimum and maximum intensity positions in convoluted red minus blue spectral plots from the at least two unique convolutions for generating a numerical skin type output. In the system and method, the physiological white light comprises three spectral intervals including a width less than 100 nanometer. The three spectral intervals pertain to red, green, and blue (RGB) colors. The three spectral intervals provide a natural white light sensation to a human eye. In the system and method, the step of comparing extreme positions of at least two unique convolutions comprises comparing a component (R-B)(W-P) for the reflected and/or re-emitted polarized light, and a component (R-B)W for the white light. The two unique convolutions in white light and polarized light further include a White Red component (WR), a White Blue component (WB), a reflected and/or re-emitted Polarized Blue component (PB) and a reflected and/or re-emitted Polarized Red component (PR). The two unique convolutions are based on a numerical value difference correlating to medical standards. The system and method may further include a spectral convolution scheme wherein multiple combinations of subtraction of blue spectrum from red, in white light and polarized white light are determined, wherein the spectral interval is expressed in a wavelength scale interval of 100 nanometers to 300 nanometers.

In an aspect of the invention, a system and method for creating a unique spectral signature of skin features may include a RGB (Red Green Blue) color channel spectral plot generated from digital images including single wavelength light matter interaction thereby generating skin type characterization output, skin moisture conductivity and skin elasticity in numerical and descriptive standards. In the system and method, the RGB (Red Green Blue) color channel spectral plots generated from digital images include multi-wavelength light matter interaction.

In an aspect of the invention, a system and method to track and store movement parameters of an imaging device moving over a subject area may include the steps of capturing an image of the subject area at a plurality of locations, identifying a direction of movement of the imaging device using an image processing technique for at least one captured frame, recognizing the direction of movement of the imaging device by comparing each frame with at least three distinct features captured to thereby triangulate a location of the imaging device, and comparing data of the captured image with a predetermined image database to store the image of the subject area and to store placement parameters of the imaging device. In the system and method, the step of capturing the image of the subject area at a plurality of locations comprises a sub step of capturing a continuous video image of the subject area. In the system and method, the step of capturing the image of the subject area at a plurality of locations comprises a sub step of capturing a frame by frame sequence of images of the subject area. In the system and method, the step of identifying a direction of movement of the imaging device using an image processing technique comprises a sub-step of a frame by frame comparison of the captured image to identify movement parameters of the imaging device. In the system and method, the step of recognizing the direction of movement of the imaging device by comparing each frame with at least three distinct features captured to triangulate a location of the imaging device comprises a sub-step of capturing a direction of movement of the imaging device by comparing three or more distinct positions across different frames.

In an aspect of the invention, an automated location tracking and data storage method and system for an imaging device may include an image capturing unit, a positioning unit coupled to the image capturing unit for positioning the imaging device on a subject area, and an image processing unit for enabling a frame by frame comparison of the captured image and for enabling the imaging device to capture three or more distinct points to triangulate a location of the imaging device to identify a direction of movement of the imaging device. In the system and method, the image capturing unit comprises a digital camera. In the system and method, the image capturing unit comprises at least one of a mobile device and a Personal Digital Assistant (PDA). In the system and method, the image processing unit comprises a comparison unit for comparing positions of three or more distinct points across different frames to capture direction of movement of the imaging device. The system and method may further include a sub-system for measuring lateral motion of the image capturing unit from a predetermined point to a new location on the subject area.

In an aspect of the invention, a system and method for determining a surgical excision margin may include illuminating a melanocytic lesion skin with an incident light source, detecting a characteristic of the light reflected and/or re-emitted from the melanocytic lesion, and determining a border between the melanocytic lesion and surrounding healthy tissue based on at least one characteristic of the reflected and/or re-emitted light. In the system and method, the incident light is directed at a selected angle alpha. In the system and method, varying alpha varies the depth of the measurement of the layers in the melanocytic lesion. Each depth has a specific angle which produces a full polarized reflection. In the system and method, the incident light is unpolarized light. The unpolarized light is at least one of white light, light of a single wavelength, and light of multiple single wavelengths. In the system and method, the incident light is polarized light. In the system and method, the reflected and/or re-emitted light is at least one of polarized light and unpolarized light. In the system and method, the characteristic is at least one of light source, light intensity, wavelength of light, angle of light, electrical and magnetic properties of the light, and polarization state of the light. An aspect of the polarization is at least one of an orientation, an amplitude, a phase, an angle, a shape, a degree, and an amount. In the system and method, determining is done using an algorithm. The algorithm involves at least one of artificial neural networks, fuzzy logic, fractal and multi-fractal analysis, non-linear regression, a genetic algorithm, white light analysis and RGB color analysis. The system and method may further include filtering the reflected and/or re-emitted light to obtain light of a wavelength defined by the filter output. Algorithmic analysis is performed on the filtered image. In the system and method, determining involves creating an image of the difference between reflected diffusion light and reflected polarized light. In the system and method, determining involves comparing the aspect of the polarization of the reflected and/or re-emitted light to a calibration signal. In the system and method, determining further comprises considering at least one of user input and a visual analysis.

Embodiments of the present invention disclose a method of managing skin health comprising capturing images of the skin, analyzing the captured images, determining at least one of susceptibility to acne and presence of acne, upon determination of the presence of acne, measuring the level of severity of acne, categorizing acne based on the measured level of severity, recommending at least one of skincare products, solutions and therapeutic regimens based on the measured level of severity of the acne, and tracking efficacy of the recommended the at least one of skincare products, solutions and therapeutic regimens.

Embodiments of the present invention disclose a method of managing acne comprising determining at least one of susceptibility to acne and presence of acne, upon determining susceptibility to acne, guesstimating the probability of occurrence of acne, upon determining presence of acne, estimating the current state of acne and guesstimating the expected state of acne, profiling the overall state of acne based on the current and expected state of acne, providing evidence-based recommendations for at least one of skincare products, solutions and therapeutic regimens based on the overall state of acne and tracking efficacy of the at least one of recommended skincare products, solutions and therapeutic regimens.

Embodiments of the present invention disclose a method of managing risk number for acne comprising determining presence of acne, upon determining presence of acne, estimating a current state of acne and guesstimating an expected state of acne, generating notifications based on an overall state of acne, determining factors controlling the risk number, determining regimens for providing recommendations for controlling the risk number based on the overall state of acne and determined factors controlling the risk number, implementing the recommended regimens, providing at least one of visual and quantitative indicators for at least one of increase and decrease in the risk number upon implementation of the recommended regimens and tracking efficacy of the recommended regimens based on the quantitative indicators for at least one of increase and decrease in the risk number.

Embodiments of the present invention a method for determining overall skin health comprising capturing one or more images of the skin illuminated with non-polarized (W) and reflected polarized (P) optical electromagnetic signals, performing spectral analysis of the one or more images acquired from the degree of polarization of at least one of reflection, absorption and re-emission of the non-polarized (W) and reflected polarized (P) optical electromagnetic signals from the skin to obtain pluralities of biophysical properties of the skin, wherein the biophysical properties are at least one of a structure, form and status of skin, and stage of at least one of: melanocyte, melanin, hemoglobin, porphyrins, tryptophan, nicotinamide adenine dinucleotide (NADH), flavin adenine dinucleotide (FAD), keratin, carotene, collagen, elastin, sebum, sebaceous gland activity, pore, sweat, sebaceous, moisture level, elasticity, luminosity, firmness, fine line, wrinkle count and stage, pore size, percent of open pores, skin elasticity, skin tension line, spot, skin color, psoriasis, allergy, red area, general skin disorder or infection, tumor, sunburn, rash, scratch, pimple, acne, stria, insect bite, itch, bleeding, injury, inflammation, photodamage, pigmentation, tone, tattoo, at least one of percent burn and burn classification, mole, nevus, aspects of a skin lesion, such as structure, color, dimensions and asymmetry, melanoma, automated follow-up of pigmented skin lesions, dermally observed disorder, cutaneous lesion, cellulite, boil, blistering disease, congenital dermal syndrome, (sub)-cutaneous mycoses, melasma, vascular condition, rosacea, spider vein, texture, skin ulcer, wound healing, post-operative tracking, melanocytic lesion, non-melanocytic lesion, basal cell carcinoma, seborrhoic keratosis, sebum (oiliness), nail and hair-related concern, and the like, generating a normalized Red (R) and Blue (B) color channel histogram for each of the one or more images, correlating the normalized Red (R) and Blue (B) color channel histograms to a wavelength scale to obtain Red (R) and Blue (B) color channel spectral plots and convoluting the spectral plots by subtracting the spectral plot for the polarized optical electromagnetic signal from the non-polarized optical electromagnetic signal for each color to generate Red (R) and Blue (B) normalized, composite color channel spectral plots and subtracting the normalized, composite Blue (B) channel spectral plot from the normalized, composite Red (R) channel spectral plot thereby resulting in generation of a spectral signature for the skin.

Embodiments of the present invention disclose a method for diagnosing acne comprising capturing one or more images of the skin illuminated with non-polarized (W) and reflected polarized (P) optical electromagnetic signals, performing spectral analysis of the one or more images acquired from the degree of polarization of at least one of reflection, absorption and re-emission of the non-polarized (W) and reflected polarized (P) optical electromagnetic signals from the skin to detect presence of at least one of acne-philic and acne-phobic genetic variants of Propionibacterium acnes based on strain-level analysis, generating a normalized Red (R) and Blue (B) color channel histogram for each of the one or more images, correlating the normalized Red (R) and Blue (B) color channel histograms to a wavelength scale to obtain Red (R) and Blue (B) color channel spectral plots and convoluting the spectral plots by subtracting the spectral plot for the polarized optical electromagnetic signal from the non-polarized optical electromagnetic signal for each color to generate Red (R) and Blue (B) normalized, composite color channel spectral plots and subtracting the normalized, composite Blue (B) channel spectral plot from the normalized, composite Red (R) channel spectral plot thereby resulting in generation of a spectral signature for the at least one of acne-philic and acne-phobic genetic variants of Propionibacterium acnes.

Embodiments of the present invention disclose a non-invasive method for diagnosing acne comprising generating optical electromagnetic signals, selectively filtering one or more wavelengths of the optical electromagnetic signals for illuminating skin, selectively filtering one or more wavelengths of the optical electromagnetic signals re-emitted from the skin owing to fluorescence, detecting a first set of wavelengths in a first range and a second set of wavelengths in a second range thereby facilitating detection of bacterial and non-bacterial acne and distinction therebetween, upon detecting presence of acne, determining level of severity of acne by performing at least one of acne segmentation based on establishment of evidence-based acne grading criteria, profiling the overall state of acne based on the level of severity of acne, providing evidence-based recommendations for at least one of skincare products, solutions and therapeutic regimens based on the level of severity of acne and tracking efficacy of the at least one of recommended skincare products, solutions and therapeutic regimens.

Embodiments of the present invention disclose a method for diagnosing acne comprising capturing one or more images of the skin illuminated with non-polarized (W) and reflected polarized (P) optical electromagnetic signals, performing spectral analysis of the one or more images acquired from the degree of polarization of at least one of reflection, absorption and re-emission of the non-polarized (W) and reflected polarized (P) optical electromagnetic signals from the skin to detect presence of at least one of acne-philic and acne-phobic genetic variants of Propionibacterium acnes based on strain-level analysis, generating a normalized Red (R) and Blue (B) color channel histogram for each of the one or more images, correlating the normalized Red (R) and Blue (B) color channel histograms to a wavelength scale to obtain Red (R) and Blue (B) color channel spectral plots and convoluting the spectral plots by subtracting the spectral plot for the polarized optical electromagnetic signal from the non-polarized optical electromagnetic signal for each color to generate Red (R) and Blue (B) normalized, composite color channel spectral plots and subtracting the normalized, composite Blue (B) channel spectral plot from the normalized, composite Red (R) channel spectral plot thereby resulting in generation of a spectral signature for the at least one of acne-philic and acne-phobic genetic variants of Propionibacterium acnes.

Embodiments of the present invention disclose a method for early prognosis of acne comprising detecting presence of at least one of acne-philic and acne-phobic genetic variants of Propionibacterium acnes using spectral signatures of the acne-philic and acne-phobic genetic variants of Propionibacterium acnes, monitoring the population of acne-phobic genetic variants of Propionibacterium acnes over a period of time to determine at least one of increase and decrease in the population thereof, upon determining at least one increase in the population of acne-philic of genetic variants of Propionibacterium acnes and decrease in the population of acne-phobic genetic variants of Propionibacterium acnes over the period of time and forecasting propensity to acne at an early stage based on at least one of the rate of increase in the population of acne-philic of genetic variants of Propionibacterium acnes and decrease in the population of acne-phobic genetic variants of Propionibacterium acnes over the period of time.

Embodiments of the present invention disclose a method for determining likelihood of treatment of acne comprising capturing one or more images of the skin illuminated with non-polarized (W) and reflected polarized (P) optical electromagnetic signals, performing spectral analysis of the one or more images acquired from the degree of polarization of at least one of reflection, absorption and re-emission of the non-polarized (W) and reflected polarized (P) optical electromagnetic signals from the skin to detect presence of at least one of families of bacteriophage, generating a normalized Red (R) and Blue (B) color channel histogram for each of the one or more images, correlating the normalized Red (R) and Blue (B) color channel histograms to a wavelength scale to obtain Red (R) and Blue (B) color channel spectral plots and convoluting the spectral plots by subtracting the spectral plot for the polarized optical electromagnetic signal from the non-polarized optical electromagnetic signal for each color to generate Red (R) and Blue (B) normalized, composite color channel spectral plots and subtracting the normalized, composite Blue (B) channel spectral plot from the normalized, composite Red (R) channel spectral plot thereby resulting in generation of a spectral signature for the at least one of families of bacteriophage.

Embodiments of the present invention disclose a method for fluorescence-based imaging of a target comprising illuminating the target with a light source emitting light of at least one wavelength or wavelength band causing at least one biomarker to fluoresce, detecting fluorescence of the at least one biomarker with an image detector, generating optical electromagnetic signals, performing spectral analysis of the one or more images acquired from the degree of polarization of at least one of reflection, absorption and re-emission of the non-polarized (W) and reflected polarized (P) optical electromagnetic signals from the skin to obtain pluralities of biophysical properties of the skin, wherein the biophysical properties are at least one of a structure, form and status of skin, and stage of at least one of: melanocyte, melanin, hemoglobin, porphyrins, tryptophan, nicotinamide adenine dinucleotide (NADH), flavin adenine dinucleotide (FAD), keratin, carotene, collagen, elastin, sebum, sebaceous gland activity, pore, sweat, sebaceous, moisture level, elasticity, luminosity, firmness, fine line, wrinkle count and stage, pore size, percent of open pores, skin elasticity, skin tension line, spot, skin color, psoriasis, allergy, red area, general skin disorder or infection, tumor, sunburn, rash, scratch, pimple, acne, stria, insect bite, itch, bleeding, injury, inflammation, photodamage, pigmentation, tone, tattoo, at least one of percent burn and burn classification, mole, nevus, aspects of a skin lesion, such as structure, color, dimensions and asymmetry, melanoma, automated follow-up of pigmented skin lesions, dermally observed disorder, cutaneous lesion, cellulite, boil, blistering disease, congenital dermal syndrome, (sub)-cutaneous mycoses, melasma, vascular condition, rosacea, spider vein, texture, skin ulcer, wound healing, post-operative tracking, melanocytic lesion, non-melanocytic lesion, basal cell carcinoma, seborrhoic keratosis, sebum (oiliness), nail and hair-related concern, and the like, generating a normalized Red (R) and Blue (B) color channel histogram for each of the one or more images, correlating the normalized Red (R) and Blue (B) color channel histograms to a wavelength scale to obtain Red (R) and Blue (B) color channel spectral plots and convoluting the spectral plots by subtracting the spectral plot for the polarized optical electromagnetic signal from the non-polarized optical electromagnetic signal for each color to generate Red (R) and Blue (B) normalized, composite color channel spectral plots and subtracting the normalized, composite Blue (B) channel spectral plot from the normalized, composite Red (R) channel spectral plot thereby resulting in generation of a spectral signature for the skin.

Embodiments of the present disclose a method for managing sun exposure of skin. The method comprises in a pre-sun exposure phase, inputting the location information of a user, and other contextual ambient information therefor, thereby facilitating determination of the UV level thereof, determining a baseline information in connection with the skin in a part of the day from at least one of midnight and dawn to noon, scanning at least one most predictably susceptible zone of one or more zones of the skin predictably susceptible to sun exposure, determining the Sun Protection Factor (SPF) level in connection with at least one of a sunscreen and sunblock for use by the user based on one or more quantifiable qualitative and quantitative parameters therefor, recommending the determined SPF level for the at least one of the sunscreen and sunblock for use by the user thereby facilitating search, identification and selection of one or more of the at least one of the sunscreen and sunblock products, solutions and regimens by the user, in an in-sun exposure phase, determining whether or not the UV index of the location is confined in at least one of one or more predetermined ranges, and reminding the user to scan the skin at one or more predetermined intervals depending upon the confinement of the determined UV index in at least one of the one or more predetermined ranges.

These and other systems, methods, objects, features, and advantages of the present invention will be apparent to those skilled in the art from the following detailed description of the preferred embodiment and the drawings. All documents mentioned herein are hereby incorporated in their entirety by reference.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1A depicts a block diagram of the system for managing and quantifying sun exposure, according to one or more embodiments;

FIG. 1B depicts a detailed block diagram of the host computing subsystem, of FIG. 1A, comprising a sun exposure management module, designed and implemented in accordance with one or more embodiments;

FIG. 2 depicts a flow diagram of the method implemented by the pre-sun exposure management sub-module, according to one or more embodiments;

FIG. 3 depicts a flow diagram of the method implemented by the in- or post-sun exposure management sub-module, according to one or more embodiments;

FIG. 4 depicts a first Two-Dimensional (2-D) coordinate system representation of increase in melanin versus the sun exposure duration in the form of a corresponding tabular representation therefor, in connection with melanin check and determination of the amount of exposure time based on natural potential of melanin, according to one or more embodiments;

FIG. 5 depicts a second Two-Dimensional (2-D) coordinate system representation of increase in redness versus time in connection with redness tracking during the day, i.e. intraday redness tracking, thereby resulting in indication of the allowable (or recommended) length of sun exposure (or the sun exposure duration), according to one or more embodiments;

FIG. 6 depicts a pictorial representation in connection with deployment of at least one of the multiple modalities of the system comprising at least one of the host computing, imaging, illumination subsystems and a combination thereof, according to one or more embodiments; and

FIG. 7 depicts a computer system that is a computing device and can be utilized in various embodiments of the present invention, according to one or more embodiments.

While the method and apparatus is described herein by way of example for several embodiments and illustrative drawings, those skilled in the art will recognize that the method and system for managing and quantifying sun exposure, is not limited to the embodiments or drawings described. It should be understood, that the drawings and detailed description thereto are not intended to limit embodiments to the particular form disclosed. Rather, the intention is to cover all modifications, equivalents and alternatives falling within the spirit and scope of the method and system for managing and quantifying sun exposure defined by the appended claims. Any headings used herein are for organizational purposes only and are not meant to limit the scope of the description or the claims. As used herein, the word “may” is used in a permissive sense (i.e., meaning having the potential to), rather than the mandatory sense (i.e., meaning must). Similarly, the words “include”, “including”, and “includes” mean including, but not limited to.

### DETAILED DESCRIPTION

In some embodiments, a system for managing and quantifying sun exposure and methods therefor are disclosed, in accordance with the principles of the present invention. Specifically, a system for managing and quantifying sun exposure via determination of one or more optimal quantitative and qualitative sun exposure management parameters based on identification, analysis, selection and measurement of one or more skin and related parameters, and recommendation of optimal quantitative and qualitative sun exposure management parameters for the skin both in the pre- and post- (or in-) sun exposure phases, and methods therefor are disclosed. For example, and in no way limiting the scope of the invention, the one or more quantitative sun exposure management parameters may be at least one of an intraday safe level (or amount) of sun exposure, a maximum duration of sun exposure tolerance, a sunscreen Sun Protection Factor (SPF) rating, frequencies of application and reapplication thereof in the pre- and post- (or in-) sun exposure phases in that order, and a combination thereof. Likewise, the one or more qualitative sun exposure management parameters may be at least one of preventive and curative therapeutic regimen comprising use of a sunscreen with a recommended SPF rating and recommended frequencies of application and reapplication thereof in the pre- and post- (or in-) sun exposure phases in that order. Still likewise, the one or more skin and related parameters may be at least one of quantitative and qualitative in nature. For example, and in no way limiting the scope of the invention, the one or more skin and related parameters may be the melanin, redness and moisture levels of the skin.

In some embodiments, the system may facilitate confirming melanin content (or level) and determining the intraday safe level (or amount) of sun exposure and maximum duration of sun exposure tolerance based on natural potential of melanin, in accordance with the principles of the present invention.

In some embodiments, the system may facilitate tracking redness level during the day, i.e. intraday redness level tracking, thereby resulting in indication of the allowable (or recommended) length of sun exposure (or the sun exposure duration), in accordance with the principles of the present invention.

In some embodiments, the system may facilitate determination of the necessary and sufficient quantity of at least one of sunscreen and sunblock, thereby facilitating avoidance of damage owing to sun exposure, in accordance with the principles of the present invention. Specifically, the system may facilitate measuring the actual quantity (or amount) of at least one of sunscreen and sunblock wore or put on or applied by a user, comparing the measured actual quantity (or amount) vis-à-vis the necessary and sufficient quantity of at least one of sunscreen and sunblock to be applied and recommending the user to apply the difference quantity of at least one of sunscreen and sunblock. More specifically, the system may facilitate optical measurement and determination of one or more quantitative and qualitative parameters related to at least one of sunscreen and sunblock in at least one of one or more formats, for instance lotion, spray, gel and other topical product, applied on the skin. For example, and in no way limiting the scope of the invention, the one or more quantitative and qualitative parameters related to at least one of sunscreen and sunblock may be at least one of the density, visual (or visually perceptible) indications (or perceptions) of the depth, color spectrum, for instance at least one of a color reflectance spectrum of sunscreen and a color absorption spectrum of sunblock, impact, SPF level (or rating) of the at least one of sunscreen and sunblock.

In some alternative embodiments, the system may facilitate determination of the necessary and sufficient quantity of at least one of sunscreen and sunblock, thereby facilitating avoidance of damage owing to sun exposure. Specifically, the system may facilitate measuring the actual quantity (or amount) of at least one of sunscreen and sunblock wore or put on or applied by a user, comparing the measured actual quantity (or amount) vis-à-vis the necessary and sufficient quantity of at least one of sunscreen and sunblock to be applied and recommending the user to apply the difference quantity of at least one of sunscreen and sunblock. More specifically, the system may facilitate spectral imaging and analysis of the skin with at least one of sunscreen and sunblock in at least one or more formats applied on the skin. For instance, many sunscreens and/or sunblock(s) may cover up the redness, and the level thereof of prior to and post application of the at least one of sunscreens and sunblock(s) on the skin may be used to determine coverage.

In some embodiments, the system may facilitate measurement of the redness level of the skin thereby facilitating avoidance of at least one of permanent and deep damage to the skin, in accordance with the principles of the present invention. Specifically, the system may facilitate tracking change in the redness level relative to time during a given day, i.e. intraday redness tracking. Of note is that fact that the redness of or in the skin is generally an inflammatory process that reduces the life of the skin cells. Upon damage owing to sun exposure, skin cells tend to peel much earlier than a normal 28 day cycle. Thus, sunburned or sunburnt skin may peel after only 7 days, instead of a normal 28 day cycle. In effect, a person's skin cycle is shortened.

In some embodiments, the system may facilitate at least one of Just-In-Time (JIT), in time and at the onset prognosis and measurement of the redness, in accordance with the principles of the present invention. Specifically, the system may facilitate detection of a point in time prior to blistering, thereby facilitating prevention of damage owing to at least one of sunburn and sun exposure. More specifically, the system may facilitate prevention of deeper damage subsequent to or owing to blistering.

Of note is the fact that sunburn and the redness owing to overexposure to sun is a function of the temperature, UV intensity, time of exposure, humidity, moisture and melanin levels of the skin, wind speed among other things. Thus, the system may facilitate measuring the aforementioned quantifiable qualitative and quantitative parameters thereby facilitating determination of the propensity to burn (or predictive susceptibility to sunburn upon sun exposure), intraday safe level (or amount) of sun exposure, maximum duration of sun exposure tolerance so as to avoid burning or causing extensive damage to the skin.

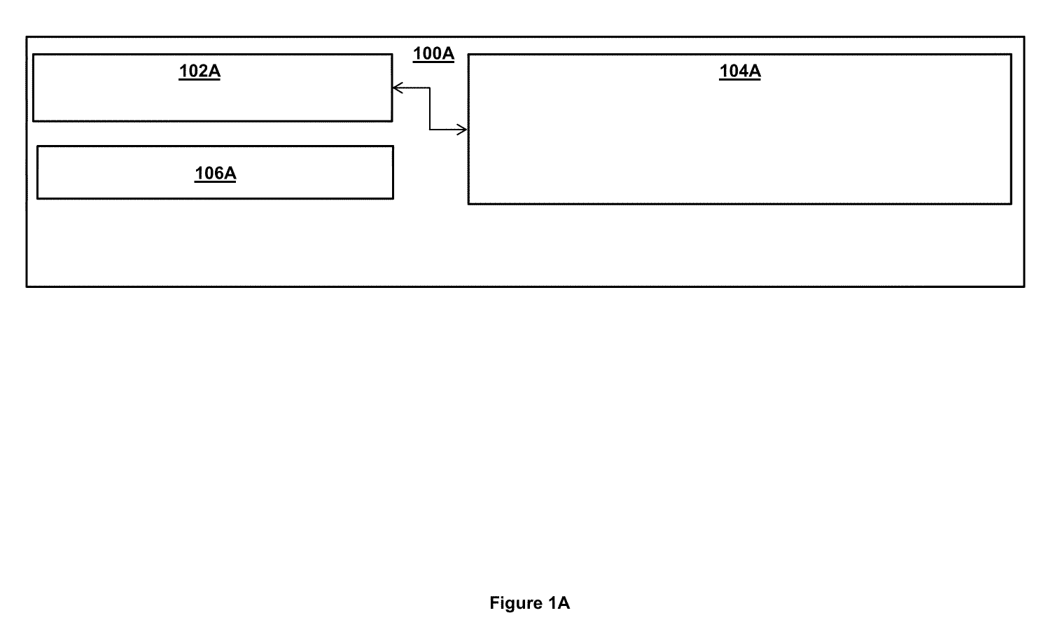

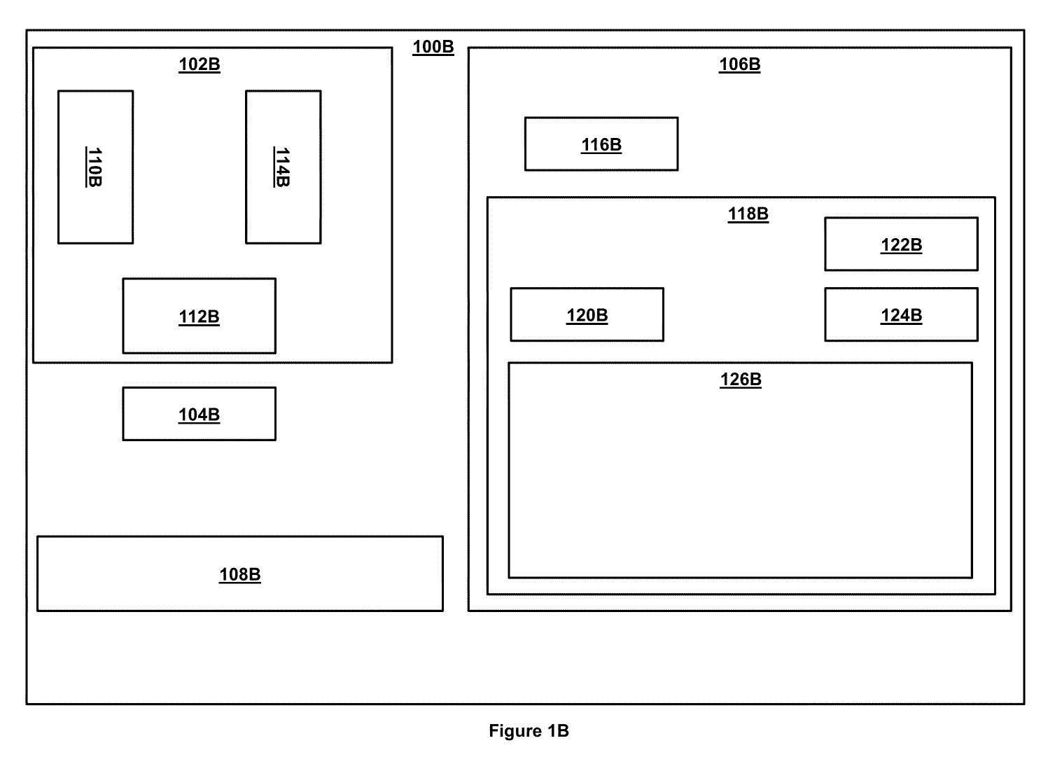

FIG. 1A depicts a block diagram of the system for managing and quantifying sun exposure, according to one or more embodiments.

The system 100A may comprise an imaging (or sensor) subsystem 102A, a host computing subsystem 104A and an illumination subsystem 106A.

The system 100A, by virtue of its design and implementation, may facilitate execution of an Opto-Magnetic method based on interaction between electromagnetic radiation and matter, for instance light-matter interaction, using digital imaging for analysis of skin subjected to sun exposure management including, but not limited to, 1) measurement of one or more optimal quantitative sun exposure management parameters, such as the intraday safe level (or amount) of sun exposure, maximum duration of sun exposure tolerance, sunscreen Sun Protection Factor (SPF) rating, frequencies of application and reapplication thereof in the pre- and post- (or in-) sun exposure phases in that order, 2) determination of one or more optimal qualitative sun exposure management parameters, such as the preventive therapeutic regimen. Further, the system 100A may facilitate recommendation of optimal measured quantitative and determined qualitative sun exposure management parameters. Specifically, the Opto-Magnetic method may facilitate generation of unique spectral signatures (or Opto-Magnetic Fingerprint or OMF) from digitally captured images of skin thereby facilitating analysis of the samples subjected to sun exposure management based on Opto-Magnetic properties of light-skin interaction. More specifically, in addition, the Opto-Magnetic method may facilitate generation of unique spectral signatures (or Opto-Magnetic Fingerprint or OMF) from digitally captured images of skin thereby facilitating analysis of the skin samples subjected to sun exposure management and quantification.

The system 100A may facilitate recommendation of the intraday safe level (or amount) of sun exposure, maximum duration of sun exposure tolerance, sunscreen Sun Protection Factor (SPF) rating, frequencies of application and reapplication thereof in the pre- and post- (or in-) sun exposure phases in that order, and the therapeutic regimen therefor based on skin imaging and analysis of the images both in the pre- and post-sun exposure phases. The system 100A may also facilitate sun exposure management including, but not limited to, imaging, analysis, monitoring, treating the skin and efficacy tracking of the recommended sunscreen products, solutions and therapeutic regimens. The system 100A may facilitate selection of an appropriate wavelength via utilization of the illumination subsystem 106A. The system 100A may facilitate search, identification, recommendation and selection of appropriate sunscreen products, solutions and therapeutic regimens based on the status of skin.

The illumination subsystem 106A may be one or more electromagnetic radiation sources. In some embodiments, the illumination subsystem 106A may be a set of Light Emitting Diodes (LEDs).

The illumination subsystem 106A may be adapted to emit polarized and unpolarized electromagnetic signals of multiple wavelengths. The polarized electromagnetic signal is angled white light, whereas the unpolarized electromagnetic signal is non-angled white light.

As depicted in FIG. 1A, in some embodiments, the imaging (or sensor) subsystem 102A may be coupled to the illumination subsystem 106A. In some other embodiments, the imaging (or sensor) subsystem 102A may comprise the illumination unit 106A. In some embodiments, the imaging (or sensor) subsystem 102A may be a portable handheld image scanner.

As depicted in FIG. 1A, the sensor subsystem 102A may be a device that converts optical images (or optical signals) to electric signals. In some embodiments, the sensor subsystem 102A may capture continuous digital images of skin. Specifically, the sensor subsystem 102A may capture continuous digital images of the skin illuminated with white light both, non-angled and angled. By way of, and by no way of limitation, the sensor subsystem 102A may be anyone selected from a group consisting of a Complementary Metal-Oxide-Semiconductor (CMOS) image sensor, Charged Coupled Device (CCD) image sensor, and the like.

Again, as depicted in FIG. 1A, the sensor subsystem 102A may be coupled to the host computing subsystem 104A.

In some embodiments, multiple modalities in connection with the design, deployment and implementation of the system are disclosed, in accordance with the principles of the present invention.

In some modalities, the system 100A may be designed, deployed and implemented as a single assembly (or compact system) (not numbered and shown herein) comprising the imaging (or sensor) subsystem 102A, host computing subsystem 104A and illumination subsystem 106A. Specifically, the imaging (or sensor) subsystem 102A, illumination subsystem 106A and host computing subsystem 104A may be fixedly operably coupled to each other thereby resulting in the realization of the single assembly (or compact system), namely a sun exposure management device (not numbered and shown herein).

In some other modalities, the system 100A may be designed, deployed and implemented as a modular system (not numbered and shown herein) comprising an imaging and illumination subsystem (not numbered and shown herein) comprising the imaging (or sensor) subsystem 102A and illumination subsystem 106A and a computing subsystem comprising the host computing subsystem 104A, wherein the imaging and illumination and computing subsystems may be wirelessly operably and communicably coupled to each other. The imaging and illumination subsystem of the modular system may comprise a microcomputer subsystem (not numbered and shown herein).

The term “digital image” refers to a representation of a two-dimensional image using ones and zeros (or binary digits or bits). The digital image may be of vector or raster type depending on whether or not the image resolution is fixed. However, without qualifications the term “digital image” usually refers to raster images.

Likewise, the term “digital imaging or digital image acquisition” refers to creation of digital images, typically from a physical object. Digital imaging or digital image acquisition may include processing, compression, storage, printing and display of digital images.

The term “digital image processing” refers to the use of computer algorithms to perform image processing on digital images. As a subfield of digital signal processing, digital image processing may have many advantages over analog image processing, for instance digital image processing may allow a much wider range of algorithms to be applied to the input data, and may avoid problems, such as the build-up of noise and signal distortion, during processing.

For example, and in no way limiting the scope of the invention, in certain embodiments, the sensor subsystem 102A may be selected on the basis of the following specifications: color is color or monochrome; optical format; horizontal pixels X vertical pixels; pixel size; one or more performance parameters, such as maximum frame rate, data rate, maximum power dissipation, quantum efficiency, dynamic range and supply voltage; output; one or more features, such as integrated Analog-to-Digital Converter (ADC) and micro lenses; and environment, such as operating temperature.

The term “image processing”, as used herein, refers to any form of signal processing for which the input is an image, such as photographs or frames of video. The output of image processing may be either an image or a set of characteristics or parameters related to the image. Most image-processing techniques involve treating the image as a two-dimensional signal and applying standard signal-processing techniques thereto.

Image processing usually refers to digital image processing, but optical and analog image processing are also possible. The acquisition of images, i.e. producing the input image in the first place, is referred to as imaging.

Medical imaging refers to the techniques and processes used to create images of the human body (or parts thereof) for clinical purposes (medical procedures seeking to reveal, diagnose or examine disease) or medical science (including the study of normal anatomy and physiology). As a discipline and in the widest sense, medical imaging is part of biological imaging and incorporates radiology (in the wider sense), radiological sciences, endoscopy, (medical) thermography, medical photography and microscopy (e.g. for human pathological investigations).

FIG. 1B depicts a detailed block diagram of the host computing subsystem, of FIG. 1A, comprising a sun exposure management module, designed and implemented in accordance with one or more embodiments.

The host computing subsystem 100B may comprise a processing unit 102B, support circuits 104B, a memory unit 106B and an Input/Output (or I/O) unit 108B respectively.

The host computing subsystem 100B, by virtue of design and implementation, facilitates performance of overall sun exposure management.

In some embodiments, the host computing subsystem 100B may be at least one of a portable computer, tablet computer, personal digital assistant (PDA), ultra mobile PC, Internet tablet, smartphone, carputer, pentop computer, and the like.

The processing unit 102B may comprise one or more commercially available microprocessors or microcontrollers that facilitate data processing and storage. The processing unit 1028 may further comprise an Arithmetic Logic Unit (or ALU) 1108, a Control Unit (or CU) 1128 and a Register Unit (or RU) 1148.

The support circuits 1048 may facilitate operation of the processing unit 1028 and include one or more clock circuits, power supplies, cache, input/output circuits, displays, and the like.

The memory unit 106B comprises at least one of Read Only Memory (ROM), Random Access Memory (RAM), disk drive storage, optical storage, removable storage and/or the like. The memory further 1068 comprises an Operating System (OS) 1168 and the sun exposure management module 1188. For example, and in no way limiting the scope of the invention, the sun exposure management module 118B may be a mobile application.

The sun exposure management module 118B comprises a pre-sun exposure management sub-module 120B, in- or post-sun exposure management sub-module 122B, melanin management sub-module 124B and a redness management sub-module 126B.

As used in general, the term “Erythema” refers to the redness of the skin or mucous membranes, caused by hyperemia of superficial capillaries. One of the many causes of the redness of the skin is solar radiation (or sunburn). In addition, the redness of the skin is a key indicator of the skin condition from the standpoint of sun exposure management. As a matter of fact, the redness of the skin is the first sign of skin damage.

The pre-sun exposure management sub-module 120B may facilitate implementation of a method comprising 1) inputting a location information of a user, and other contextual ambient information therefor, thereby facilitating determination of the UV level thereof, 2) determining a baseline information in connection with the skin of the user in a part of the day from at least one of midnight and dawn to noon via measuring at least one of the melanin, redness (or Erythema), moisture levels and a combination thereof, in at least one of susceptible (or prone), user-anticipated, and a combination thereof, one or more zones of sun exposure on the skin by scanning the skin, 3) rescanning at least one zone, of the at least one of susceptible (or prone), user-anticipated, and a combination thereof, one or more zones of sun exposure on the skin, which is at least one of most susceptible (or prone), most anticipated, and a combination thereof, 4) determining the SPF level of a given sunscreen for use by the user based on one or more at least one of intrinsic and extrinsic, and at least one of quantifiable qualitative and quantitative parameters therefor, and 5) recommending the SPF level of a given sunscreen for use by the user thereby facilitating the user in search, identification and selection of one or more sunscreen products, solutions and therapeutic regimens.

The in- or post-sun exposure management sub-module 122B may facilitate implementation of a method comprising 1) at least one of determining whether or not the UV index or level of the location is confined in at least one of one or more predetermined ranges, and 2) reminding the user to scan the skin at one or more predetermined intervals, i.e. time period or duration, corresponding to or depending upon the confinement of the determined UV index or level in at least one of the one or more predetermined ranges.

The melanin management sub-module 124B may facilitate confirming melanin content and determining safe level (or amount) of sun exposure and maximum duration of sun exposure tolerance based on natural potential of melanin.

The redness management sub-module 126B may facilitate redness tracking during the day, i.e. intraday redness tracking, thereby resulting in indication of the allowable (or recommended) length of sun exposure (or the sun exposure duration). Based on the above fact that the redness of the skin is the key indicator of the skin condition from the standpoint of sun exposure management. Specifically, based on the fact that the redness of the skin is the first sign of skin damage. In operation, the redness management sub-module 126B may facilitate detection or recognition of the onset of the redness of skin, thereby facilitating users in precisely determining the safe level (or amount) of sun exposure and maximum duration of sun exposure tolerance, for instance when the users need to stop avoiding sun exposure, and the potential intensity of damage in the event that the users exceed or postpone the maximum duration of sun exposure tolerance. In some scenarios, in the event that the users have the redness level that is abnormal, for instance owing rosacea, then the abnormal redness level serves as an indicator of damage in the skin.

In some embodiments, the redness management sub-module may facilitate measurement of at least one of permanent, deep damage to the skin and a combination thereof. Specifically, the redness of the skin is generally an inflammatory process that reduces the life of the skin cells. Upon damage owing to sun exposure, the skin cells tend to peel much earlier than a normal 28 day cycle. Thus, sunburned skin may peel after only 7 days, instead of the normal 28 day cycle. In effect, a person's skin cycle is shortened.

In addition, the measurement of the redness of the skin in advance or at an early stage may facilitate determination of at least one of a point in time, threshold and safe level prior to blistering owing to sunburn, thereby preventing permanent damage. Further, in addition, the measurement of the redness of the skin in advance or at an early stage may facilitate deeper damage caused by blistering.

In some embodiments, the method implemented by the pre-sun exposure management sub-module is disclosed in detail, according to one or more embodiments.

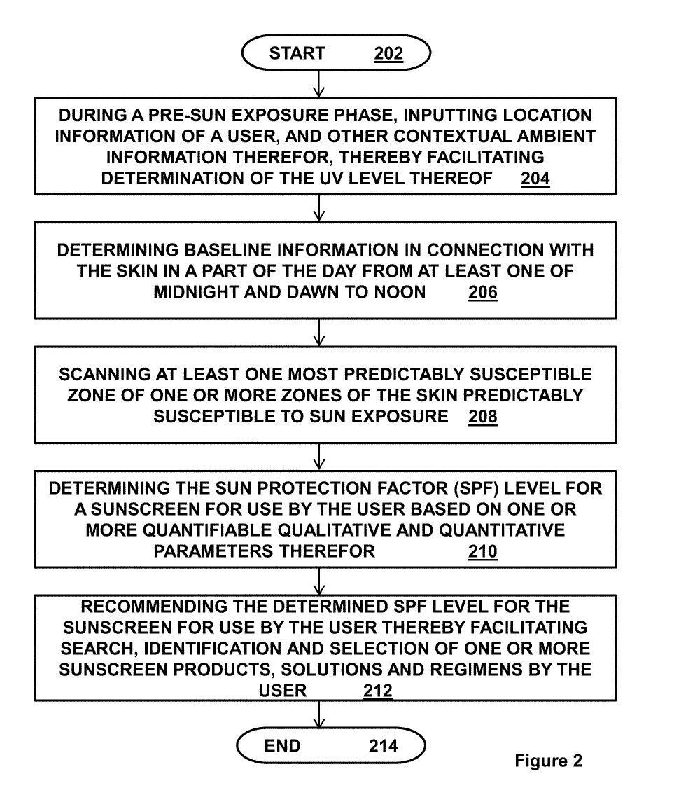

FIG. 2 depicts a flow diagram of the method implemented by the pre-sun exposure management sub-module, according to one or more embodiments.

The method 200 starts at step 202 and proceeds to step 204.

At step 204, the method 200 comprises in a pre-sun exposure phase, inputting location information of a user, and other contextual ambient information therefor, thereby facilitating determination of the UV level thereof. For example, and in no way limiting the scope of the invention, the location information may comprise geographic latitude information. In some scenarios, the geographic latitude information may comprise geographic data of a location, such as a Zone Improvement Plan (ZIP) or postal code, street address, etc., thereby facilitating determination of the associated geographic coordinates (often expressed as latitude and longitude) of the location based on the concept of geocoding using the geographic data. For example, in so far as the effect of the geographic latitude information/data on the UV radiation exposure is concerned, the UV energy above 42 degrees north latitude, i.e. a line approximately between the northern border of California and Boston, is insufficient for cutaneous vitamin D synthesis from November through February, whereas in far northern latitudes, the reduced intensity of the UV energy lasts for up to 6 months. On the contrary, in the United States, latitudes below 34 degrees north, i.e. a line between Los Angeles and Columbia, S.C., allow for cutaneous production of vitamin D throughout the year.

In some embodiments, one or more factors affecting UV radiation exposure including, but not limited to, season, time of day, cloud cover, smog, skin melanin content, sunscreen, may also be taken into consideration. For example, in so far as the effect of cloud cover on the UV radiation exposure is concerned, complete cloud cover reduces UV energy by 50%, whereas shade, including that produced by severe pollution, reduces the UV energy by 60%.

Further, in so far as the effect of intraday alteration in the UV level on the UV radiation exposure is concerned, the composition of UV radiation changes throughout the day. For example, at high noon, the UV radiation reaching ground level is 95% UVA and 5% UVB, whereas before 10 AM and after 2 PM the percentage of the UV radiation reaching ground level changes over time to 99% UVA and 1% UVB. The change in percentage of the UV radiation reaching ground level is caused by the reflection of UVB rays back into space due to sun angle as the earth slowly rotates on the Earth's axis of rotation. The rate of change is faster the farther the position is away from the equator, more north or south. On average over a day, 98.7% of the UV radiation that reaches the Earth's surface is UVA. UVC is almost completely absorbed by the ozone layer and, thus fails to penetrate the atmosphere in any appreciable quantities.

At step 206, the method 200 comprises determining baseline information in connection with the skin in a part of the day from at least one of midnight and dawn to noon. For example, the part of the day may be morning. Specifically, the method 200 comprises measuring at least one of the melanin, redness (or Erythema), moisture levels and a combination thereof, in at least one of susceptible (or prone), user-anticipated, and a combination thereof, one or more zones of sun exposure on the skin, by a user via deployment of the system. Stated differently, the method 200 comprises measuring at least one of the melanin, redness (or Erythema), moisture levels and a combination thereof, in one or more zones of the skin at least one of susceptible (or prone) to, anticipated to be subjected to, and a combination thereof, to sun exposure, by a user via deployment of the system. For example, and in no way limiting the scope of the invention, the one or more zones may be at least one of the inner forearm, forehead, back, other zones and a combination thereof. For example, the measuring at least one of the melanin, redness (or Erythema), moisture levels and a combination thereof, comprises capturing one or more images of the inner forearm, forehead, back, other zones and a combination thereof. Notable here is the fact that the inner forearm (or forearms) provides a more accurate view of natural melanin level of a user as the inner forearm is at least one of less and not exposed to the sun, while other body parts typically show results of long term exposure.

At step 208, the method 200 comprises scanning at least one zone, of the at least one of susceptible (or prone), user-anticipated, and a combination thereof, one or more zones of sun exposure on the skin, which is at least one of most susceptible (or prone), most anticipated, and a combination thereof, by the user via deployment of the system. For example, and in no way limiting the scope of the invention, the forehead and back are typically the most susceptible to sun exposure. Notable here is the fact that in general persons having exposed melanin have greater resistance to sun damage, and hence stay longer in the sun in those parts.

As used in medicine, the term “baseline” refers to information found at the beginning of a study or other initial known value, which is used for comparison with later data. The concept of a baseline is essential to the daily practice of medicine in order to establish a relative rather than absolute meaning to data. The meaning of baseline in medicine is very similar to that of the running baseline (baseball) being the direct path that a base runner is taking to the base he/she is in route to. If the base runner is outside the 3 foot margin around his running baseline, then he/she is considered out. Whereas in the event that a patient with kidney failure, i.e. whose creatinine is usually 3.0 mg/dL, suddenly has a creatinine of 5.0 mg/dL, then the creatinine of the patient is out of normal. For that person with kidney failure, absolute normal no longer applies because the patient will never again be able to obtain an absolutely normal creatinine level (0.5-1.2 mg/dL) with kidneys that no longer function properly.

At step 208, the method 200 comprises determining Sun Protection Factor (SPF) level of the user based on one or more at least one of intrinsic and extrinsic, and at least one of quantifiable qualitative and quantitative parameters therefor. For example, and in no way limiting the scope of the invention, the at least one of intrinsic and extrinsic, and at least one of quantifiable qualitative and quantitative parameters are at least one of the skin melanin content, redness (or Erythema), moisture levels, UV level, one or more independent variables therefor, and a combination thereof.

At step 210, the method 200 comprises recommending a SPF level for the user thereby facilitating the user in search, identification and selection of one or more skincare products, solutions and regimens.

The method 200 ends at step 212.

In some embodiments, the method implemented by the in- or post-sun exposure management sub-module is disclosed in detail, according to one or more embodiments.

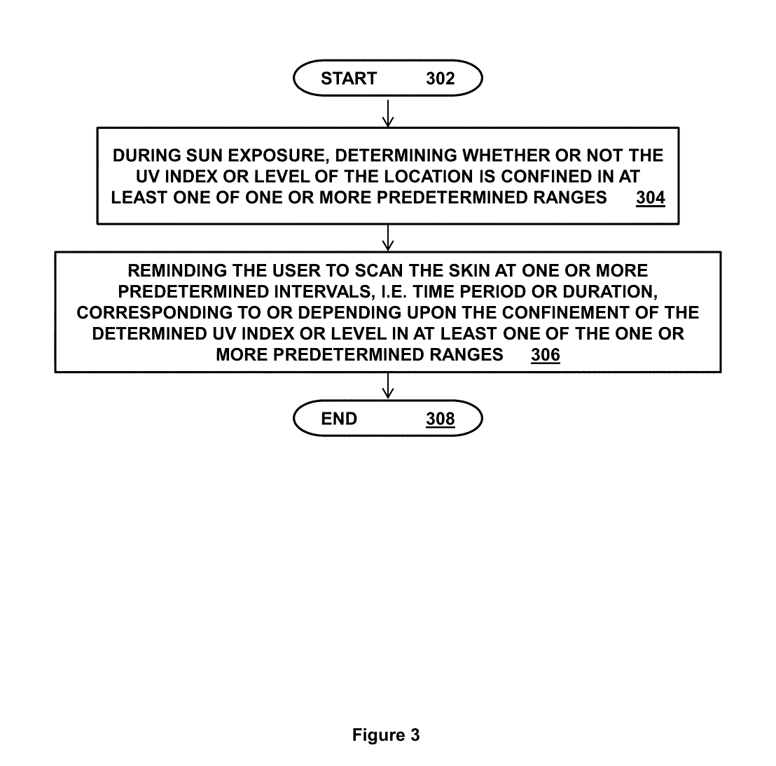

FIG. 3 depicts a flow diagram of the method implemented by the in- or post-sun exposure management sub-module, according to one or more embodiments.

The method 300 starts at step 302 and proceeds to step 304.

At step 304, the method 300 comprises in sun exposure, determining whether or not the UV index or level of the location is confined in at least one of one or more predetermined ranges. For example, and in no way limiting the scope of the invention, the UV index or level in a first interval (or predetermined range) may exceed a minimum value of approximately 7, whereas the UV index or level in a second interval (or predetermined range) may be below a maximum value of approximately 6.

At step 306, the method 300 comprises reminding the user to scan the skin at one or more predetermined intervals, i.e. time period or duration, corresponding to or depending upon the confinement of the determined UV index or level in at least one of the one or more predetermined ranges. For example, and in no way limiting the scope of the invention, the user is reminded via at least one of a vibration (or audio alert or warning) generated by the system and notification generated (or initiated) by a mobile application implemented by the system. Specifically, the user is reminded to conduct a scan of the skin at least one of every Fifteen (15) minutes in the event that the UV index or level is greater than (>) 7 and every Sixty (60) minutes in the event that the UV index or level is less than (>) 6.

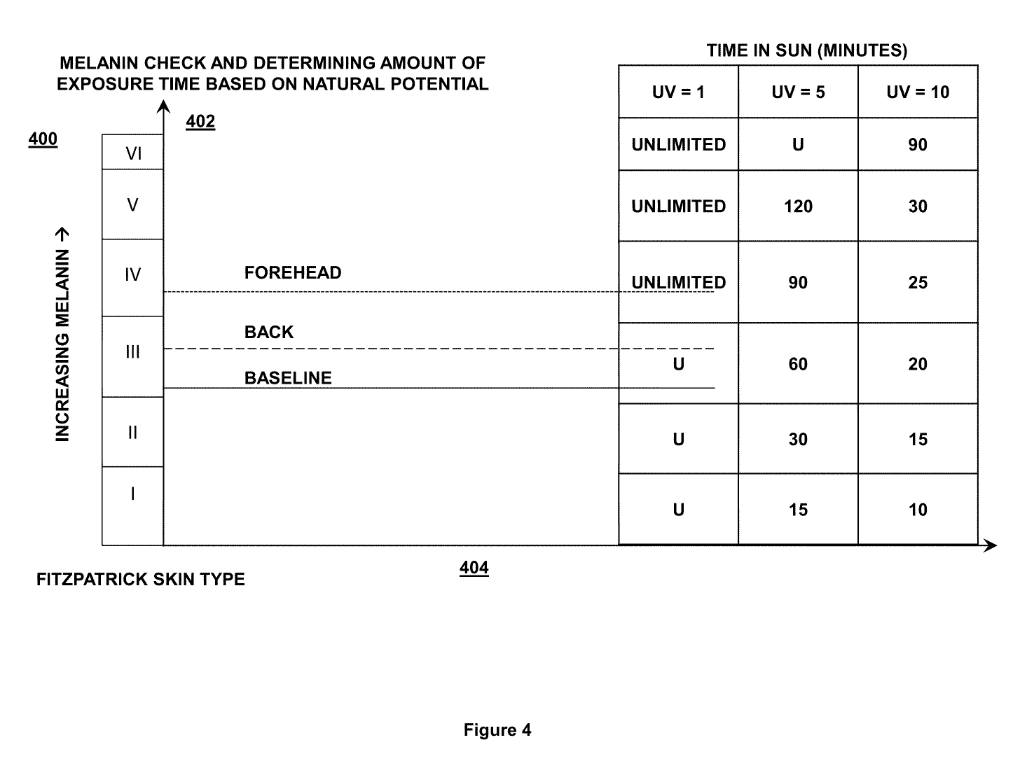

FIG. 4 depicts a first Two-Dimensional (2-D) coordinate system representation of increase in melanin versus the sun exposure duration in the form of a corresponding tabular representation therefor, in connection with melanin check and determination of the amount of exposure time based on natural potential of melanin, according to one or more embodiments.

As used herein and in general, the term “Fitzpatrick Scale (or Fitzpatrick skin typing test or Fitzpatrick phototyping scale” refers to a numerical classification schema for the color of skin. The Fitzpatrick Scale serves as a way to classify the response of different types of skin, i.e. Fitzpatrick skin type, to UV light. In accordance with the Fitzpatrick Scale, the skin is divided into one or more following types, namely 1) Type I (scores 0-6) comprises pale white; blond or red hair; blue eyes; freckles, which always burns, but never tans; 2) Type II (scores 7-13) comprises white; fair; blond or red hair; blue, green or hazel eyes, which usually burns, but tans minimally; 3) Type III (scores 14-20) comprises cream white; fair with any hair or eye color; quite common, which sometimes mild burn, but tans uniformly; 4) Type IV (scores 21-27) comprises moderate brown; typical Mediterranean skin tone, which rarely burns, but always tans well; 5) Type V (scores 28-34) comprises dark brown; Middle Eastern skin types, which very rarely burns, but tans very easily; and 7) Type VI (scores 35+) comprises deeply pigmented dark brown to black, which never burns, but tans very easily.

As depicted in FIG. 4, the vertical Y-axis 402 of the first Two-Dimensional (2-D) coordinate system representation 400 comprises at least seven distinct skin types, and correspondingly increasing melanin content in that order. The horizontal X-axis 404 of the first Two-Dimensional (2-D) coordinate system representation 400 comprises the sun exposure duration, for instance in minutes, in the form of a Table 1, which is a corresponding tabular representation therefor.

| UV = 1 | UV = 5 | UV = 10 |
|---|---|---|
| UNLIMITED | U | 90 MINUTES |
| UNLIMITED | 120 MINUTES | 30 MINUTES |
| UNLIMITED | 90 MINUTES | 25 MINUTES |
| U | 60 MINUTES | 20 MINUTES |
| U | 30 MINUTES | 15 MINUTES |
| U | 15 MINUTES | 10 MINUTES |

Table 1 depicts a tabular representation of the sun exposure duration in minutes in connection with the Fitzpatrick skin types corresponding to (or against) one or more distinct UV indexes or levels.

In some embodiments, the Table 1 may be a proxy for time in the sun, i.e. the sun exposure duration in minutes, before onset of inflammation or redness. Depending on the UV exposure, the Table 1 may be tabulated or tabularized based on at least one of one or more continuous and discrete functions for determining the exposure.

In some embodiments, a similar table may be used in connection with the maximum time allowed before severe burning.

In some scenarios, the Table 1 may be subjected to continuous refinement based on the feedbacks of the users in the form of measurements and questionnaires facilitating determination of the maximum time for sun exposure based on the given or available or current conditions.

In some scenarios, the Table 1 may be continuously adapted based on actual UV conditions during a given day.

The Table 1 may be capable of defining the frequency of scans necessary to ensure that the user may proactively avoid the onset of redness.

As depicted in FIG. 4, for example, the forehead belonging to the Fitzpatrick skin type IV, i.e. Type IV (scores 21-27) comprises moderate brown; typical Mediterranean skin tone, which rarely burns, but always tans well, possesses the following corresponding sun exposure durations of “UNLIMITED,” 90 and 25 minutes based on the three distinct UV indexes or levels, namely UV=1, UV=5 and UV=10 in that order.

Likewise, for example, the back belonging to the Fitzpatrick skin type III, i.e. Type III (scores 14-20) comprises cream white; fair with any hair or eye color; quite common, which sometimes mild burn, but tans uniformly, possesses the following corresponding sun exposure durations of “U,” 60 and 20 minutes based on the three distinct UV indexes or levels, namely UV=1, UV=5 and UV=10 in that order.

Still likewise, for example, the baseline for the skin type III possesses the following corresponding sun exposure durations of “U,” 60 and 20 minutes based on the three distinct UV indexes or levels, namely UV=1, UV=5 and UV=10 in that order.

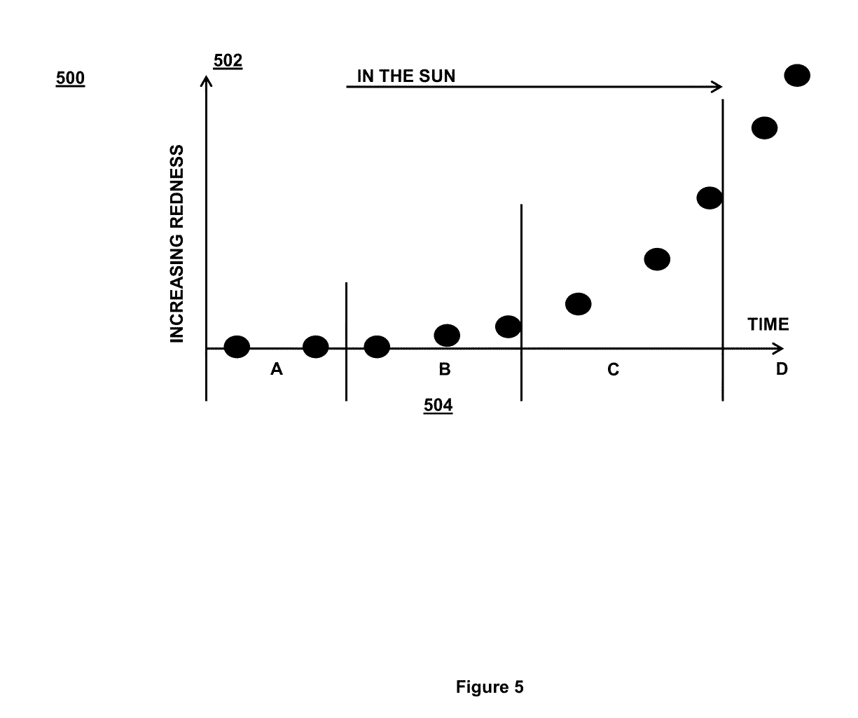

FIG. 5 depicts a second Two-Dimensional (2-D) coordinate system representation of increase in redness versus time in connection with redness tracking during the day, i.e. intraday redness tracking, thereby resulting in indication of the allowable (or recommended) length of sun exposure (or the sun exposure duration), according to one or more embodiments.

As depicted in FIG. 5, the vertical Y-axis 502 of the second Two-Dimensional (2-D) coordinate system representation 500 comprises one or more values corresponding to increase in the redness. The horizontal X-axis 504 of the Two-Dimensional (2-D) coordinate system representation 500 comprises one or more distinct phases of sun exposure marked as corresponding zones with distinct color labels. For example, and in no way limiting the scope of the invention, and for purposes of clarity and expediency the phases have been hereinafter named as the “PHASE A OR MORNING OR PRE-SUN EXPOSURE” marked correspondingly as “GREEN ZONE,” “PHASE B OR MAIN SUN EXPOSURE” marked correspondingly as “GREEN ZONE,” “PHASE C OR MODERATE RISK” marked correspondingly as “YELLOW ZONE,” and “PHASE D OR VERY HIGH RISK” marked correspondingly as “RED ZONE” respectively.

More specifically, the “PHASE A OR MORNING OR/PRE-SUN EXPOSURE” phase marked correspondingly as “GREEN ZONE” comprises a phase, wherein the redness of the skin is at least one of non-existent and at a minimal level. The “PHASE A OR MORNING OR/PRE-SUN EXPOSURE” phase is the most optimal phase for determination of baseline measurements of the skin. Likewise, the “PHASE B OR MAIN SUN EXPOSURE” phase marked correspondingly as “GREEN ZONE” comprises a phase, wherein slight onset of the redness occurs in the skin. The “PHASE B OR MAIN SUN EXPOSURE” phase is still fine for the skin, before damage accumulates. Still likewise, the “PHASE C OR MODERATE RISK” marked correspondingly as “YELLOW ZONE” comprises a phase, wherein increased redness of the skin occurs that may lead to damage. Specifically, in the “PHASE C OR MODERATE RISK” damage starts, however the damage is not enough to cause peeling or burns. Further, the “PHASE D OR VERY HIGH RISK” marked correspondingly as “RED ZONE” comprises a phase, wherein the person is recommended to avoid further exposure to sun, or otherwise risks severe burns and permanent skin damage.

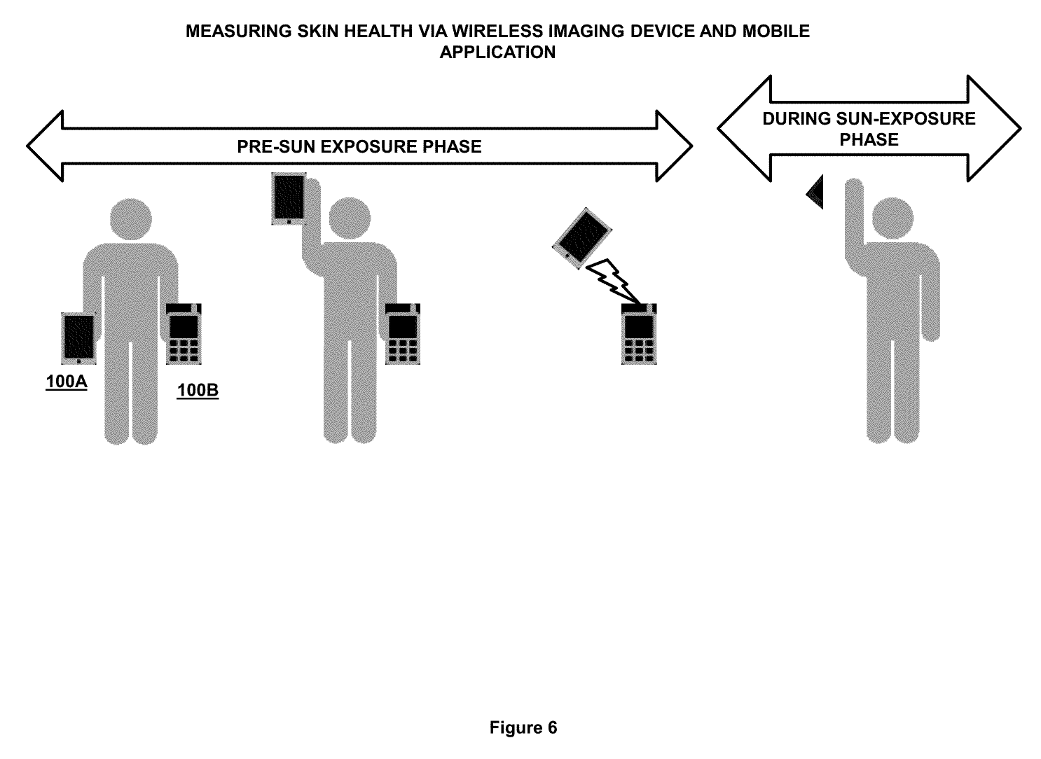

FIG. 6 depicts a pictorial representation in connection with deployment of at least one of the multiple modalities of the system comprising at least one of the host computing, imaging, illumination subsystems and a combination thereof, according to one or more embodiments.

In some operational embodiments involving deployment of the modular system, a user may activate the sun exposure management module 118B, for instance a mobile application, installed on the host computing subsystem 100B, as depicted in FIG. 1B, (or the host computing subsystem 104A, as depicted in FIG. 1A,) in a pre-sun exposure phase, for instance in at least one of morning and prior to sun exposure. The user may scan using the sun exposure management device, as disclosed in one of the modalities of the system 100A, of FIG. 1A, the one or more zones on the skin thereby facilitating determining baseline information in connection with the skin via measurement of the melanin, redness, moisture levels, and other characteristics of the skin. In some scenarios, the sun exposure management device, as disclosed in one of the modalities of the system 100A, of FIG. 1A, may be wirelessly communicably and operably coupled to the host computing subsystem 100B, of FIG. 1B. In some embodiments, upon determining baseline information, the sun exposure management device may automatically synchronize with the host computing subsystem thereby facilitating synchronization of one or more data, for instance the benchmark settings, thresholds, alerts and frequency table, therebetween. In some embodiments, the sun exposure management device may facilitate algorithmic processing thereby facilitating independent use of the same.

In some scenarios, the user may optionally scan the skin using the sun exposure management device without the intervention of the sun exposure management module 118B installed on the host computing subsystem 100B, of FIG. 1B. The sun exposure management device may be preset to scan everytime the user activates. In some scenarios, in the event that the redness level is above a threshold, the user may receive from the sun exposure management device a color coded response, for instance in at least one of green, yellow and red, based on the redness level relative to the threshold. In some scenarios, in the event that the user may be at least one of under high risk of burning and actually burning, the sun exposure management device may also vibrate.

In some embodiments, the system may be coupled to and synchronized with one or more medical monitors (or health monitoring devices), for instance IWATCH™ or UV bands, thereby facilitating detecting actual UV exposure in real time, or any other connected devices, which are smart wide area and short range devices that have the benefit of connecting to a network, including, but not limited to, remote sensors, remote monitoring, actuating devices, associated aggregation devices, PCs, laptops, tablets, eReaders, mobile handsets, femto cells, routers. In some embodiments, the system may be coupled to and synchronized with skin health monitoring devices. In some scenarios, the system may be used by multiple users, for instance a family, to track using a single device or multiple devices.

Advantageously, in some embodiments, the system 100 may facilitate determining at least one of safe level (or amount) of sun exposure, maximum duration of sun exposure tolerance and a combination thereof, based on detection of the onset of the redness of the skin as opposed to only guesstimation of the time limit. Specifically, the system 100 overcomes the problems in connection with the determination of at least one of safe level (or amount) of sun exposure, maximum duration of sun exposure tolerance and a combination thereof based only on the guesstimation of the time limit via deployment of at least one of a tabular representation of the UV indices and conventional standards. This gives a personalized advice on how long you can stay in the sun based on melanin levels (your actual amount), moisture (measuring the effects of wind and environment), and actually redness. At the first onset of redness, the system knows how long until the severity worsens and the damage gets severe and irreversible.

Advantageously, in some embodiments, the system 100 may facilitate enhanced estimation of the maximum duration of sun exposure tolerance, for instance the time to spend in the sun or time to sun exposure, via measurement of the actual melanin level as opposed to an indirect estimation of the time.

Advantageously, in some embodiments, the system 100 may facilitate dynamic adjustment of the maximum duration of sun exposure tolerance via measurement of at least one of the actual melanin and redness levels as opposed to deployment of the Fitzpatrick questionnaires, wherein a user may fail to accurately respond to each of the queries in the questionnaires therein.

In some advantageous embodiments, the system 100 may facilitate determination of a preventive therapeutic regimen in connection with a given sunscreen based on one or more skin parameters. Specifically, the system 100 may facilitate determination of an actual time to reapply the given sunscreen based on actual measurements of the one or more skin parameters as opposed to at least one of estimated and average measurements, for instance as in UVEBAND. For example, and in no way limiting the scope of the invention, the system 100 facilitates determination of the actual time to reapply the given sunscreen based on actual measurements of the moisture, melanin, redness levels, time elapsed in UV exposure or duration of UV exposure.

Still, in some advantageous embodiments, the system 100 may facilitate determination of the type of acne and assessment of the condition, for instance the level of severity thereof, thereby facilitating determination of the at least one of safe level (or amount) of sun exposure, maximum duration of sun exposure tolerance and a combination thereof based on the determined acne type and assessed severity level thereof. Of note here is the fact that the UV directly aggravates the acne, and the condition thereof, thus overexposure may render pores to worsen.

In yet other advantageous embodiments involving deployment of the modular system, upon determination of the redness, melanin, and moisture levels of the skin of the user using the sun exposure management module 118B installed on the host computing subsystem 100B, of FIG. 1B, the settings may be transferred wirelessly to the sun exposure management device so that the user may be exempted from using the sun exposure management module, thereby facilitating the user to merely use signals from the sun exposure management device to determine the redness, melanin, and moisture levels of the skin.

In some scenarios, at least one of the sun exposure management device and the sun exposure management module, for instance the mobile application, may be coupled to at least one of a UV detection band and UV service, thereby facilitating determination intraday, i.e. during the day, active exposure to UV as opposed to estimation of the same.

In some embodiments, quantification of the amount of sunscreen on the skin using optical measurement of one or more parameters of the sunscreen is disclosed, in accordance with the principles of the present invention. Specifically, the system facilitates quantifying amount of sunscreen on the skin thereby facilitating determination of whether or not the sunscreen is used in excess. More specifically, the system facilitates optically measuring one or more parameters, for instance the density of the sunscreen, visual indications of depth, color spectrum and effects thereof based on the condition of skin in at least one of absence of the sunscreen and different levels of the sunscreen.

In some embodiments, quantification of the amount of sunscreen on the skin using the system based on spectral or color analysis of the skin with the sunscreen applied thereupon is disclosed, in accordance with the principles of the present invention. In some embodiments, the system facilitates determining the level of redness at least one of pre and post application of the sunscreen on the skin, thereby facilitating the determination of the coverage of the sunscreen.

In some embodiments, the system may facilitate determining the propensity to sunburn, at least one of the safe level (or amount) of sun exposure, maximum duration of sun exposure tolerance and a combination thereof, so as to at least one of avoid burning and causing extensive damage to the skin.

In some embodiments, the system may facilitate comprehensively combining the natural level of melanin, current level therefor and redness level of users.

In some embodiments, the system facilitates linking the hemoglobin, melanin and keratin levels thereby facilitating determining the at least one of the safe level (or amount) of sun exposure, maximum duration of sun exposure tolerance and a combination thereof, for instance the actual time to stay in the sun and to exit. In addition, the system may facilitate measuring the redness and melanin levels in the skin to ensure overexposure to sun.

### Example Computer System

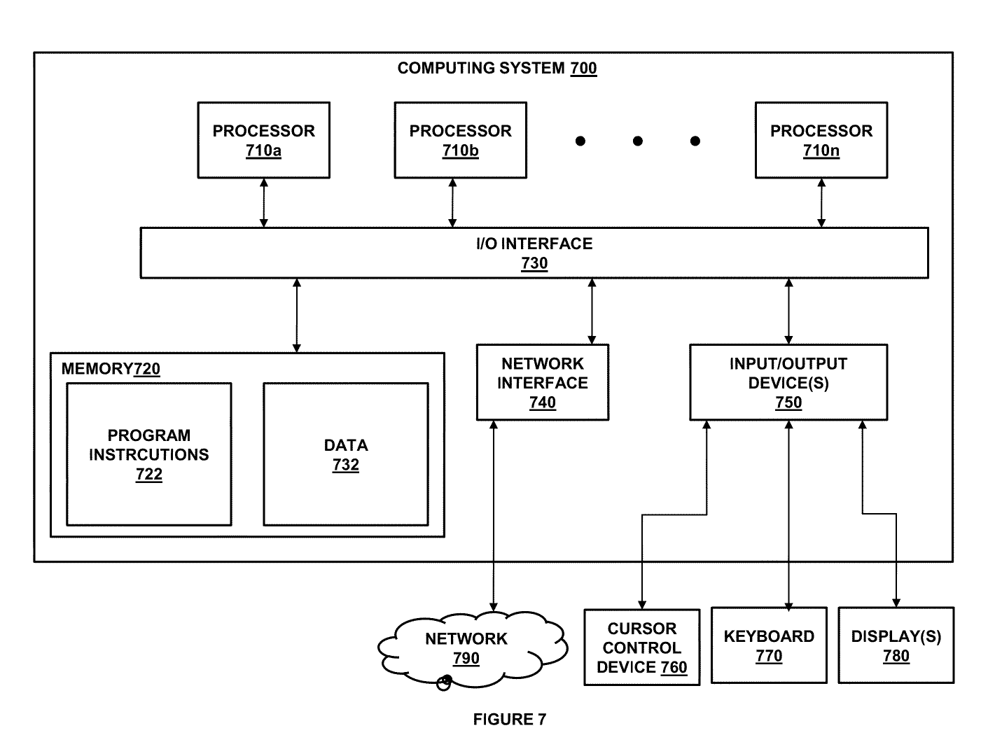

FIG. 7 depicts a computer system that is a computing device and can be utilized in various embodiments of the present invention, according to one or more embodiments.

Various embodiments of method and apparatus for managing and quantifying sun exposure, as described herein, may be executed on one or more computer systems, which may interact with various other devices. One such computer system is computer system 700 illustrated by FIG. 7, which may in various embodiments implement any of the elements or functionality illustrated in FIGS. 1A, 1B and 2-6. In various embodiments, computer system 700 may be configured to implement methods described above. The computer system 700 may be used to implement any other system, device, element, functionality or method of the above-described embodiments. In the illustrated embodiments, computer system 700 may be configured to implement methods 200 and 300, as processor-executable executable program instructions 722 (e.g., program instructions executable by processor(s) 710A-N) in various embodiments.

In the illustrated embodiment, computer system 700 includes one or more processors 710a-n coupled to a system memory 720 via an input/output (I/O) interface 730. The computer system 700 further includes a network interface 740 coupled to I/O interface 730, and one or more input/output devices 750, such as cursor control device 760, keyboard 770, and display(s) 780. In various embodiments, any of components may be utilized by the system to receive user input described above. In various embodiments, a user interface (e.g., user interface) may be generated and displayed on display 780. In some cases, it is contemplated that embodiments may be implemented using a single instance of computer system 700, while in other embodiments multiple such systems, or multiple nodes making up computer system 700, may be configured to host different portions or instances of various embodiments. For example, in one embodiment some elements may be implemented via one or more nodes of computer system 700 that are distinct from those nodes implementing other elements. In another example, multiple nodes may implement computer system 700 in a distributed manner.

In different embodiments, computer system 700 may be any of various types of devices, including, but not limited to, a personal computer system, desktop computer, laptop, notebook, or netbook computer, mainframe computer system, handheld computer, workstation, network computer, a camera, a set top box, a mobile device, a consumer device, video game console, handheld video game device, application server, storage device, a peripheral device such as a switch, modem, router, or in general any type of computing or electronic device.

In various embodiments, computer system 700 may be a uniprocessor system including one processor 710, or a multiprocessor system including several processors 710 (e.g., two, four, eight, or another suitable number). Processors 710a-n may be any suitable processor capable of executing instructions. For example, in various embodiments processors 710 may be general-purpose or embedded processors implementing any of a variety of instruction set architectures (ISAs), such as the x96, POWERPC®, SPARC®, or MIPS® ISAs, or any other suitable ISA. In multiprocessor systems, each of processors 710a-n may commonly, but not necessarily, implement the same ISA.

System memory 720 may be configured to store program instructions 722 and/or data 732 accessible by processor 710. In various embodiments, system memory 720 may be implemented using any suitable memory technology, such as static random access memory (SRAM), synchronous dynamic RAM (SDRAM), nonvolatile/Flash-type memory, or any other type of memory. In the illustrated embodiment, program instructions and data implementing any of the elements of the embodiments described above may be stored within system memory 720. In other embodiments, program instructions and/or data may be received, sent or stored upon different types of computer-accessible media or on similar media separate from system memory 720 or computer system 700.

In one embodiment, I/O interface 730 may be configured to coordinate I/O traffic between processor 710, system memory 720, and any peripheral devices in the device, including network interface 740 or other peripheral interfaces, such as input/output devices 750. In some embodiments, I/O interface 730 may perform any necessary protocol, timing or other data transformations to convert data signals from one components (e.g., system memory 720) into a format suitable for use by another component (e.g., processor 710). In some embodiments, I/O interface 730 may include support for devices attached through various types of peripheral buses, such as a variant of the Peripheral Component Interconnect (PCI) bus standard or the Universal Serial Bus (USB) standard, for example. In some embodiments, the function of I/O interface 730 may be split into two or more separate components, such as a north bridge and a south bridge, for example. Also, in some embodiments some or all of the functionality of I/O interface 730, such as an interface to system memory 720, may be incorporated directly into processor 710.

Network interface 740 may be configured to allow data to be exchanged between computer system 700 and other devices attached to a network (e.g., network 790), such as one or more external systems or between nodes of computer system 700. In various embodiments, network 790 may include one or more networks including but not limited to Local Area Networks (LANs) (e.g., an Ethernet or corporate network), Wide Area Networks (WANs) (e.g., the Internet), wireless data networks, some other electronic data network, or some combination thereof. In various embodiments, network interface 740 may support communication via wired or wireless general data networks, such as any suitable type of Ethernet network, for example; via telecommunications/telephony networks such as analog voice networks or digital fiber communications networks; via storage area networks such as Fiber Channel SANs, or via any other suitable type of network and/or protocol.

Input/output devices 750 may, in some embodiments, include one or more display terminals, keyboards, keypads, touchpads, scanning devices, voice or optical recognition devices, or any other devices suitable for entering or accessing data by one or more computer systems 700. Multiple input/output devices 750 may be present in computer system 700 or may be distributed on various nodes of computer system 700. In some embodiments, similar input/output devices may be separate from computer system 700 and may interact with one or more nodes of computer system 700 through a wired or wireless connection, such as over network interface 740.

In some embodiments, the illustrated computer system may implement any of the methods described above, such as the methods illustrated by the flowcharts of FIGS. 2 and 3. In other embodiments, different elements and data may be included.

Those skilled in the art will appreciate that computer system 700 is merely illustrative and is not intended to limit the scope of embodiments. In particular, the computer system and devices may include any combination of hardware or software that can perform the indicated functions of various embodiments, including computers, network devices, Internet appliances, PDAs, wireless phones, pagers, etc. Computer system 700 may also be connected to other devices that are not illustrated, or instead may operate as a stand-alone system. In addition, the functionality provided by the illustrated components may in some embodiments be combined in fewer components or distributed in additional components. Similarly, in some embodiments, the functionality of some of the illustrated components may not be provided and/or other additional functionality may be available.

Those skilled in the art will also appreciate that, while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 700 may be transmitted to computer system 700 via transmission media or signals such as electrical, electromagnetic, or digital signals, conveyed via a communication medium such as a network and/or a wireless link. Various embodiments may further include receiving, sending or storing instructions and/or data implemented in accordance with the foregoing description upon a computer-accessible medium or via a communication medium. In general, a computer-accessible medium may include a storage medium or memory medium such as magnetic or optical media, e.g., disk or DVD/CD-ROM, volatile or non-volatile media such as RAM (e.g., SDRAM, DDR, RDRAM, SRAM, etc.), ROM, etc.

The methods described herein may be implemented in software, hardware, or a combination thereof, in different embodiments. In addition, the order of methods may be changed, and various elements may be added, reordered, combined, omitted, modified, etc. All examples described herein are presented in a non-limiting manner. Various modifications and changes may be made as would be obvious to a person skilled in the art having benefit of this disclosure. Realizations in accordance with embodiments have been described in the context of particular embodiments. These embodiments are meant to be illustrative and not limiting. Many variations, modifications, additions, and improvements are possible. Accordingly, plural instances may be provided for components described herein as a single instance. Boundaries between various components, operations and data stores are somewhat arbitrary, and particular operations are illustrated in the context of specific illustrative configurations. Other allocations of functionality are envisioned and may fall within the scope of claims that follow. Finally, structures and functionality presented as discrete components in the example configurations may be implemented as a combined structure or component. These and other variations, modifications, additions, and improvements may fall within the scope of embodiments as defined in the claims that follow.

While the foregoing is directed to embodiments of the present invention, other and further embodiments of the invention may be devised without departing from the basic scope thereof, and the scope thereof is determined by the claims that follow.

1. A method for managing sun exposure of skin comprising:
in a pre-sun exposure phase, inputting the location information of a user, and other contextual ambient information therefor, thereby facilitating determination of the UV level thereof;
determining a baseline information in connection with the skin in a part of the day from at least one of midnight and dawn to noon;
scanning at least one most predictably susceptible zone of one or more zones of the skin predictably susceptible to sun exposure;
determining the Sun Protection Factor (SPF) level in connection with at least one of a sunscreen and sunblock for use by the user based on one or more quantifiable qualitative and quantitative parameters therefor;
recommending the determined SPF level for the at least one of the sunscreen and sunblock for use by the user thereby facilitating search, identification and selection of one or more of the at least one of the sunscreen and sunblock products, solutions and regimens by the user;
in an in-sun exposure phase, determining whether or not the UV index of the location is confined in at least one of one or more predetermined ranges; and
reminding the user to scan the skin at one or more predetermined intervals depending upon the confinement of the determined UV index in at least one of the one or more predetermined ranges.

2. The method of claim 1, wherein the quantifiable qualitative and quantitative parameters are at least one of the temperature, wind speed, UV intensity, duration of exposure, moisture, humidity, melanin, redness levels of the skin and a combination thereof.

3. The method of claim 1, wherein the step of determining the SPF level for the at least one of sunscreen and sunblock for use by the user further comprises measuring the quantifiable qualitative and quantitative parameters.

4. The method of claim 3, wherein the measurement of the redness level of the skin facilitates avoidance of at least one of permanent and deep damage to the skin.

5. The method of claim 1, further comprises determining the necessary and sufficient quantity of at least one of sunscreen and sunblock, thereby facilitating avoidance of damage owing to sun exposure.

6. The method of claim 5, wherein determining the necessary and sufficient quantity of at least one of sunscreen and sunblock further comprise measuring the actual quantity of at least one of sunscreen and sunblock applied by a user, comparing the measured actual quantity vis-à-vis the necessary and sufficient quantity of at least one of sunscreen and sunblock to be applied and recommending the user to apply the difference quantity of at least one of sunscreen and sunblock.

7. The method of claim 6, wherein the measuring the actual quantity of the at least one of sunscreen and sunblock applied by the user comprises optical measurement and determination of one or more quantitative and qualitative parameters related to the at least one of sunscreen and sunblock in at least one of one or more formats applied on the skin.

8. The method of claim 7, wherein the one or more quantitative and qualitative parameters related to the at least one of sunscreen and sunblock is at least one of the density, visual indications of the depth, color spectrum, impact, SPF level (or rating) of the at least one of sunscreen and sunblock.

9. The method of claim 8, wherein the color spectrum is at least one of a color reflectance spectrum of the sunscreen and a color absorption spectrum of the sunblock.

10. The method of claim 9, wherein the measuring the actual quantity of the at least one of sunscreen and sunblock applied by the user comprises spectral imaging and analysis of the skin with at least one of sunscreen and sunblock in at least one or more formats applied on the skin.

11. The method of claim 10, wherein the at least one of sunscreen and sunblock covers up the redness such that the redness level thereof prior to and post application of the at least one of sunscreen and sunblock on the skin is used to determine coverage.

12. The method of claim 1, further comprises measuring the redness level of the skin thereby facilitating avoidance of at least one of permanent and deep damage to the skin.

13. The method of claim 12, the measuring the redness level of the skin further comprises tracking change in the redness level relative to time during a given day.

14. The method of claim 1, further comprises confirming natural melanin level in the skin, and determining the intraday safe level of sun exposure and maximum duration of sun exposure tolerance based on natural melanin level.
